(19) United States
(12) Patent Application Publication
(10) Pub. No.: US 2024/0058037 A1
(43) Pub. Date: Feb. 22, 2024

(54) EXTERNAL FIXATION DEVICES, SYSTEMS, AND METHODS

(71) Applicant: XFix8 LLC, Keller, TX (US)

(72) Inventors: Michael MEIGGS, Keller, TX (US); Daniel J. TRIPLETT, Smithfield, UT (US)

(21) Appl. No.: 18/235,296

(22) Filed: Aug. 17, 2023

Related U.S. Application Data
(60) Provisional application No. 63/398,739, filed on Aug. 17, 2022.

(51) Int. Cl. A61B 17/64

(52) U.S. Cl. CPC A61B 17/6466

(57) ABSTRACT

An external fixation system may have a first bone anchoring body configured to be secured to a first bone, a second bone anchoring body configured to be secured to a second bone, and a first strut assembly. The first strut assembly may have a first end configured to be removably coupled to the first bone anchoring body, a second end configured to be removably coupled to the second bone anchoring body, an intermediate portion that extends between the first end and the second end, the intermediate portion having a length that is adjustable, and a clutch mechanism. The clutch mechanism may be movable between an unlocked position in which the clutch mechanism allows both increase and reduction in the length, and a restricted position in which the clutch mechanism allows one of increase and reduction in the length while restricting the other of increase and reduction in the length.

### CROSS-REFERENCE TO RELATED APPLICATIONS

This application claims the benefit of U.S. Provisional Application Ser. No. 63/398,739, filed on Aug. 17, 2022 and entitled “External Fixation Devices, Systems, and Methods,” the disclosure of which is incorporated by reference as though set forth herein in its entirety.

### TECHNICAL FIELD

The present disclosure relates to devices, systems, and methods for external fixation. More specifically, the present disclosure relates to devices, systems, and methods for providing external fixation to unstable, broken, or fractured joints/bones.

### BACKGROUND

The present disclosure relates to systems, devices, and methods for external fixation of bones/joints. External fixation can be used for long or short term stabilization of traumatic injuries, joint injuries, limb-lengthening procedures, etc.

The devices, systems, and methods described herein may be utilized for stabilization of a traumatic injury until a long-term stabilization device can be applied. Short-term or temporary stabilization may allow soft tissues to recover from initial trauma prior to a more definitive skeletal fixation procedure. For example, short-term or temporary stabilization may allow for reduction of swelling, healing of open wounds, and/or healing of skin abrasions prior to an open reduction internal fixation procedure. External fixation may also be useful during transportation from one site to another, or for periods of time when appropriate trauma care may not be available. For example, short-term external fixation may be appropriate in battlefield situations, field hospital situations, etc.

### SUMMARY

The various systems and methods of the present disclosure have been developed in response to the present state of the art, and in particular, in response to the problems and needs in the art that have not yet been fully solved by currently available external fixation systems and methods. In some embodiments, the external fixation systems of the present disclosure may provide enhanced adjustability, speed of implementation, and/or patient comfort.

In some embodiments, an external fixation system may have a first bone anchoring body configured to be secured to a first bone, a second bone anchoring body configured to be secured to a second bone, and a first strut assembly. The first strut assembly may have a first end configured to be removably coupled to the first bone anchoring body, a second end configured to be removably coupled to the second bone anchoring body, an intermediate portion that extends between the first end and the second end, the intermediate portion having a length that is adjustable, and a clutch mechanism. The clutch mechanism may be movable between an unlocked position in which the clutch mechanism allows both increase and reduction in the length, and a restricted position in which the clutch mechanism allows one of increase and reduction in the length while restricting the other of increase and reduction in the length.

The external fixation system of any preceding paragraph may further have a first strut attachment assembly that is attachable to the first bone anchoring body and to the first end of the first strut assembly, and a second strut attachment assembly that is attachable to the second bone anchoring body and to the second end of the first strut assembly.

In the external fixation system of any preceding paragraph, the first end of the first strut assembly may have a first sphere portion. The second end of the first strut assembly comprises a second sphere portion. The first bone anchoring body may have a first arm. The second bone anchoring body may have a second arm. The first strut attachment assembly may have a first fastener configured to secure the first strut attachment assembly to the first arm, and a first strut clamp assembly with a first clamp having a first inner clamp surface configured to receive and be tightened around the first sphere portion. The second strut attachment assembly may have a second fastener configured to secure the second strut attachment assembly to the second arm, and a second strut clamp assembly with a second clamp having a second inner clamp surface configured to receive and be tightened around the second sphere portion.

In the external fixation system of any preceding paragraph, the first strut clamp assembly may be securable to the first arm by actuating only the first fastener. The second strut clamp assembly may be securable to the second arm by actuating only the second fastener.

In the external fixation system of any preceding paragraph, the first bone anchoring body may have a pin clamp body with a plurality of pin lock collets, each of which is configured to receive a bone pin and lock the bone pin in place relative to the pin clamp body.

In the external fixation system of any preceding paragraph, the first strut assembly may further have an adjustment mechanism operable independently of the clutch mechanism to adjust a length of the intermediate portion.

In the external fixation system of any preceding paragraph, the adjustment mechanism may have an adjustment knob rotatably coupled to the inner strut tube. The adjustment knob may have an inner adjustment knob thread configured to engage the outer strut tube thread to move the outer strut tube axially along the inner strut tube in response to rotation of the adjustment knob.

In the external fixation system of any preceding paragraph, the clutch mechanism may further be movable to a locked position in which the clutch mechanism does not allow variation in the length.

In the external fixation system of any preceding paragraph, the intermediate portion may have an outer strut tube, and an inner strut tube that is slidable within the outer strut tube to adjust the length. The clutch mechanism may have an adjustment collar with an inner chamfered surface, a plurality of clutch balls, a plurality of clutch springs configured to retain the clutch balls between the inner chamfered surface and an outer surface of an inner strut tube, and a clutch knob that is rotatable to move the clutch balls in relation to the inner chamfered surface such that, in the restricted position, the inner chamfered surface drives the clutch balls against the outer surface of the inner strut tube in response to reduction in the length, and in the unlocked position, the inner chamfered surface does not drive the clutch balls against the outer surface of the inner strut tube in response to reduction in the length.

The external fixation system of any preceding paragraph may further have an auxiliary bone anchoring body with an auxiliary base that is securable at an adjustable location along the length of the intermediate portion, and an auxiliary pin guide that is polyaxially adjustable relative to the auxiliary base such that the auxiliary pin guide is securable to the first bone at any of a plurality of orientations relative to the auxiliary base.

The external fixation system of any preceding paragraph may further have a second strut assembly with a third end configured to be removably coupled to the first bone anchoring body, a fourth end configured to be removably coupled to the second bone anchoring body, and a second intermediate portion that extends between the third end and the fourth end. The second intermediate portion may have a second length that is adjustable.

According to some embodiments, an external fixation system may be used to secure a first bone of a patient relative to a second bone of the patient. The external fixation system may have a first bone anchoring body configured to be secured to the first bone, a second bone anchoring body configured to be secured to the second bone, and a first strut assembly. The first strut assembly may have a first end configured to be removably coupled to the first bone anchoring body, a second end configured to be removably coupled to the second bone anchoring body, an intermediate portion that extends between the first end and the second end, the intermediate portion having a length that is adjustable, an adjustment mechanism operable to adjust a length of the intermediate portion, and a clutch mechanism operable independently of the adjustment mechanism. The clutch mechanism may be movable between an unlocked position in which the clutch mechanism allows variation in the length, and a locked position in which the clutch mechanism does not allow variation in the length.

The external fixation system of any preceding paragraph may further have a first strut attachment assembly that is attachable to the first bone anchoring body and to the first end of the first strut assembly, and a second strut attachment assembly that is attachable to the second bone anchoring body and to the second end of the first strut assembly. The first end of the first strut assembly may have a first sphere portion. The second end of the first strut assembly may have a second sphere portion. The first bone anchoring body may have a first arm. The second bone anchoring body may have a second arm. The first strut attachment assembly may have a first fastener configured to secure the first strut attachment assembly to the first arm, and a first strut clamp assembly comprising a first clamp comprising a first inner clamp surface configured to receive and be tightened around the first sphere portion. The second strut attachment assembly may have a second fastener configured to secure the second strut attachment assembly to the second arm, and a second strut clamp assembly comprising a second clamp comprising a second inner clamp surface configured to receive and be tightened around the second sphere portion. The first strut clamp assembly may be securable to the first arm by actuating only the first fastener. The second strut clamp assembly may be securable to the second arm by actuating only the second fastener.

In the external fixation system of any preceding paragraph, the first bone anchoring body may have a pin clamp body with a plurality of pin lock collets, each of which is configured to receive a bone pin and lock the bone pin in place relative to the pin clamp body.

In the external fixation system of any preceding paragraph, the intermediate portion may have an outer strut tube with an outer strut tube thread, and an inner strut tube that is slidable within the outer strut tube to adjust the length. The adjustment mechanism may have an adjustment knob rotatably coupled to the inner strut tube. The adjustment knob may have an inner adjustment knob thread configured to engage the outer strut tube thread to move the outer strut tube axially along the inner strut tube in response to rotation of the adjustment knob.

In the external fixation system of any preceding paragraph, the clutch mechanism may further be movable to a restricted position in which the clutch mechanism allows one of increase and reduction in the length while restricting the other of increase and reduction in the length.

In the external fixation system of any preceding paragraph, the intermediate portion may have an outer strut tube and an inner strut tube that is slidable within the outer strut tube to adjust the length. The clutch mechanism may have an adjustment collar with an inner chamfered surface, a plurality of clutch balls, a plurality of clutch springs configured to retain the clutch balls between the inner chamfered surface and an outer surface of an inner strut tube, and a clutch knob that is rotatable to move the clutch balls in relation to the inner chamfered surface such that, in the restricted position, the inner chamfered surface drives the clutch balls against the outer surface of the inner strut tube in response to reduction in the length, and in the unlocked position, the inner chamfered surface does not drive the clutch balls against the outer surface of the inner strut tube in response to reduction in the length.

According to some embodiments, an external fixation system may be used for securing a first bone of a patient relative to a second bone of the patient. The external fixation system may have a plurality of bone anchoring bodies, each of which is configured to be secured to at least one of the first bone and the second bone. The plurality of bone anchoring bodies may include a first bone anchoring body having a first shape, and a second bone anchoring body having a second shape different from the first shape. The external fixation system may further include a strut assembly having a variable length, and a plurality of strut attachment assemblies, each of which is attachable to any of the bone anchoring bodies and to the strut assembly to secure any of the bone anchoring bodies to the strut assembly.

The external fixation system of any preceding paragraph may further have a first strut attachment assembly that is attachable to the first bone anchoring body and to the first end of the first strut assembly, and a second strut attachment assembly that is attachable to the second bone anchoring body and to the second end of the first strut assembly. The first end of the first strut assembly may have a first sphere portion. The second end of the first strut assembly may have a second sphere portion. The first bone anchoring body may have a first arm. The second bone anchoring body may have a second arm. The first strut attachment assembly may have a first fastener configured to secure the first strut attachment assembly to the first arm, and a first strut clamp assembly with a first clamp with a first inner clamp surface configured to receive and be tightened around the first sphere portion. The second strut attachment assembly may include a second fastener configured to secure the second strut attachment assembly to the second arm, and a second strut clamp assembly with a second clamp with a second inner clamp surface configured to receive and be tightened around the second sphere portion. The first strut clamp assembly may be securable to the first arm by actuating only the first fastener. The second strut clamp assembly may be securable to the second arm by actuating only the second fastener.

In the external fixation system of any preceding paragraph, the first bone anchoring body may have a pin clamp body with a plurality of pin lock collets, each of which is configured to receive a bone pin and lock the bone pin in place relative to the pin clamp body.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1 is a perspective view of an external fixation system with two strut assemblies, according to an embodiment of the present disclosure;

FIG. 2 is an alternative perspective view of the external fixation system of FIG. 1;

FIG. 3 is an enlarged perspective view of one end of the external fixation system of FIG. 1;

FIG. 4 is an exploded perspective view of the two strut clamping assemblies shown in FIG. 3;

FIG. 5 is an exploded perspective view of one of the strut clamping assemblies shown in FIG. 4;

FIG. 6A is a front elevation view of the strut clamping assembly of FIG. 5, after assembly;

FIG. 6B is a front elevation, section view of the strut clamping assembly of FIG. 6A;

FIG. 7 is a perspective view of a strut assembly of the external fixation system shown in FIG. 1;

FIG. 8 is an exploded, perspective view of the strut assembly of FIG. 7;

FIG. 9A is an exploded, perspective view of the clutch mechanism shown in FIG. 8;

FIG. 9B is a perspective view of the clutch mechanism of FIG. 9A, after assembly;

FIG. 10A is a side elevation view of the strut assembly of FIG. 7;

FIG. 10B is a side elevation, section view of the strut assembly of FIG. 10A;

FIG. 10C is an enlarged view of the clutch mechanism of the section view of FIG. 10B, in a first configuration;

FIG. 10D is an enlarged view of the clutch mechanism of the section view of FIG. 10B, in a second configuration;

FIG. 10E is an enlarged view of the clutch mechanism of the section view of FIG. 10B, in a third configuration;

FIG. 10F is an enlarged view of the locking mechanism of the section view of FIG. 10B;

FIG. 10G is an enlarged view of the adjustment mechanism of the section view of FIG. 10B;

FIG. 11 is a front elevation view of the strut assembly of FIG. 7;

FIG. 12 is a perspective view of an external fixation system spanning an ankle joint, according to an embodiment of the present disclosure;

FIG. 13 is an alternative perspective view of the external fixation system of FIG. 12;

FIG. 14 is another alternative perspective front view of the external fixation system of FIG. 12 in isolation;

FIG. 15 is a perspective view of the ankle pin clamp assembly of the external fixation assembly of FIG. 12;

FIG. 16 is an exploded, perspective view of the ankle pin clamp assembly of FIG. 15;

FIG. 17 is an exploded, perspective view of the auxiliary pin clamp assembly of the external fixation assembly of FIG. 12;

FIG. 18A is an exploded, perspective view of a “T-shaped” pin clamp body, according to an embodiment of the present disclosure;

FIG. 18B is a perspective view of the T-shaped pin clamp body of FIG. 18A, after assembly;

FIG. 18C is a side elevation, section view of the T-shaped pin clamp body of FIG. 18B;

FIG. 19A is an exploded, perspective view of an “X-shaped” or “t-shaped” pin clamp body, according to another embodiment of the present disclosure;

FIG. 19B is a perspective view of the “X-shaped” pin clamp body of FIG. 19A, after assembly;

FIG. 20 is a perspective view of a “t-shaped” pin clamp body, according to another embodiment of the present disclosure;

FIG. 21 is a perspective view of an external fixation system spanning a knee joint, according to an embodiment of the present disclosure;

FIG. 22 is a perspective view of a two-level external fixation system spanning a knee joint and an ankle joint, according to an embodiment of the present disclosure;

FIG. 23 is a perspective view of the ankle pin clamp assembly of FIG. 15 coupled to an ankle of a patient, and the X-shaped pin clamp body of FIG. 19A coupled to a tibia of the patient;

FIG. 24 is a perspective view of a first strut assembly coupled with the system shown in FIG. 23;

FIG. 25 is a perspective view of the auxiliary pin clamp assembly of FIG. 17 coupled to the first strut assembly shown in FIG. 24;

FIG. 26 is a perspective view of a second strut assembly coupled with the system shown in FIG. 25;

FIG. 27 is a perspective view of one end of the strut assembly of FIG. 7 being coupled to the T-shaped pin clamp of FIG. 18A, in a modular fashion;

FIG. 28 is a perspective view of two T-shaped pin clamps of FIG. 18A coupled to a femur and a tibia of a patient;

FIG. 29 is a perspective view of a first strut assembly coupled with the system shown in FIG. 28; and

FIG. 30 is a perspective view of a second strut assembly coupled with the system shown in FIG. 29.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to external fixation devices, systems, and methods. Those skilled in the art will recognize that the following description is merely illustrative of the principles of the technology, which may be applied in various ways to provide many alternative embodiments. The present disclosure illustrates external fixation of knee and ankle joints for the purposes of illustrating the concepts of the present design. However, it will be understood that other variations and uses are contemplated including, but not limited to, applications in the arm, wrist, finger, toe, spine, pelvis, any other bone or joint, etc.

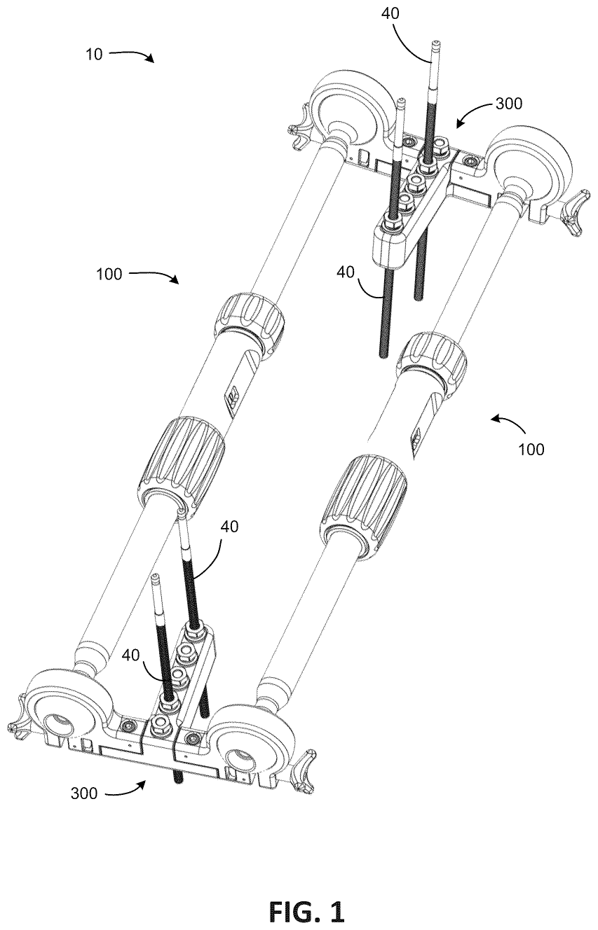

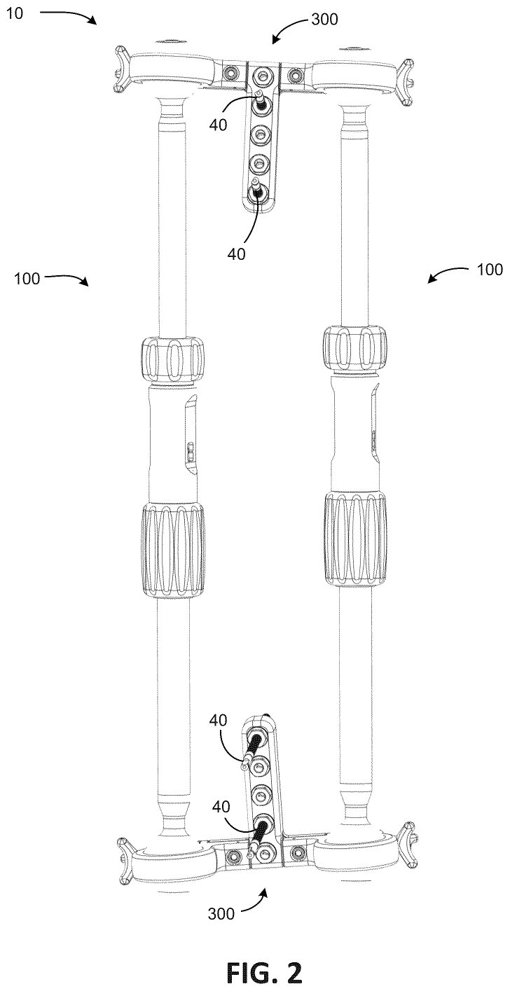

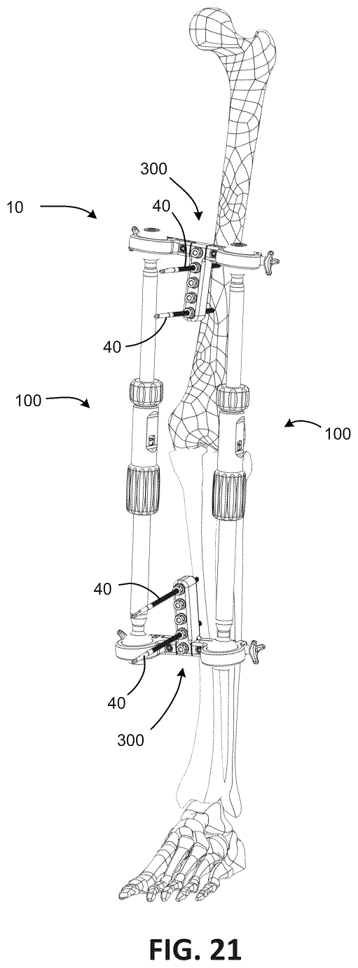

FIGS. 1 and 2 show two views of an external fixation system 10 with two strut assemblies, according to an embodiment of the present disclosure. FIG. 1 is a perspective view of the external fixation system 10, and FIG. 2 is a top view of the external fixation system 10. The external fixation system 10 may be utilized for temporary fixation of a wide variety of bone structures and/or joints. In some embodiments, the external fixation system 10 may be used to span and stabilize a knee joint, as will be discussed in more detail below with respect to FIGS. 21 and 28-30.

The external fixation system 10 may generally include a strut assembly 100, a pin clamp body 300, and a fixation pin 40. More particularly, the external fixation system 10 may include two strut assemblies 100, two pin clamp bodies 300, and a set of fixation pins 40.

In some embodiments, each strut assembly 100, pin clamp body 300, and fixation pin 40 of the external fixation systems disclosed herein may comprise the same/similar parts that may be reproduced to help reduce manufacturing costs. For example, the strut assemblies 100 of the external fixation system 10 shown in FIGS. 1 and 2 may be substantially identical to each other in construction and parts that are utilized.

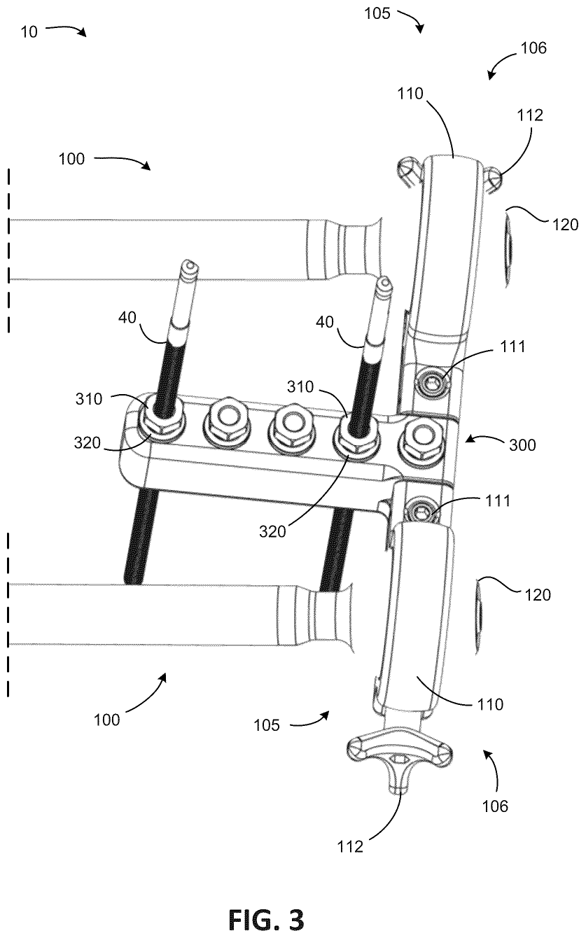

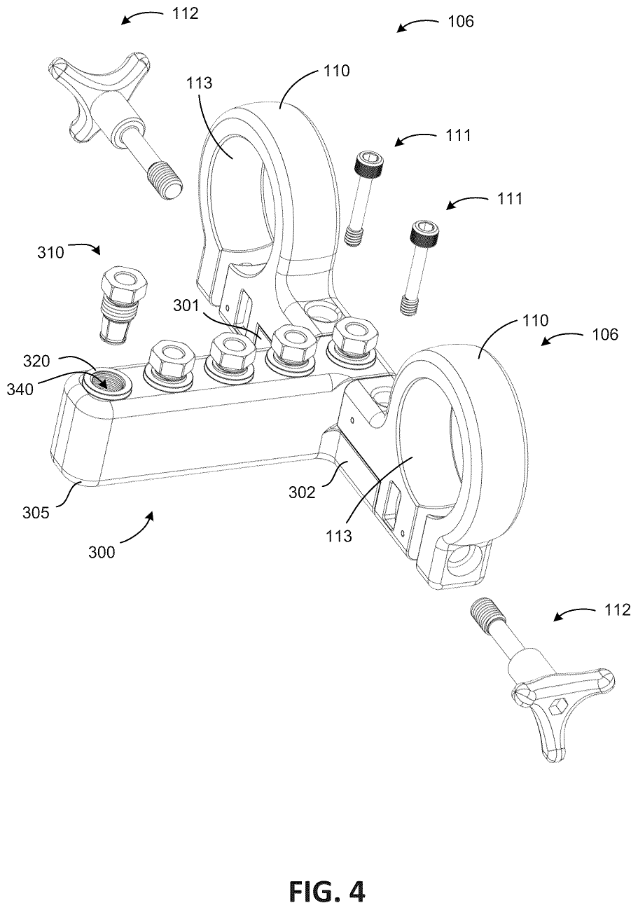

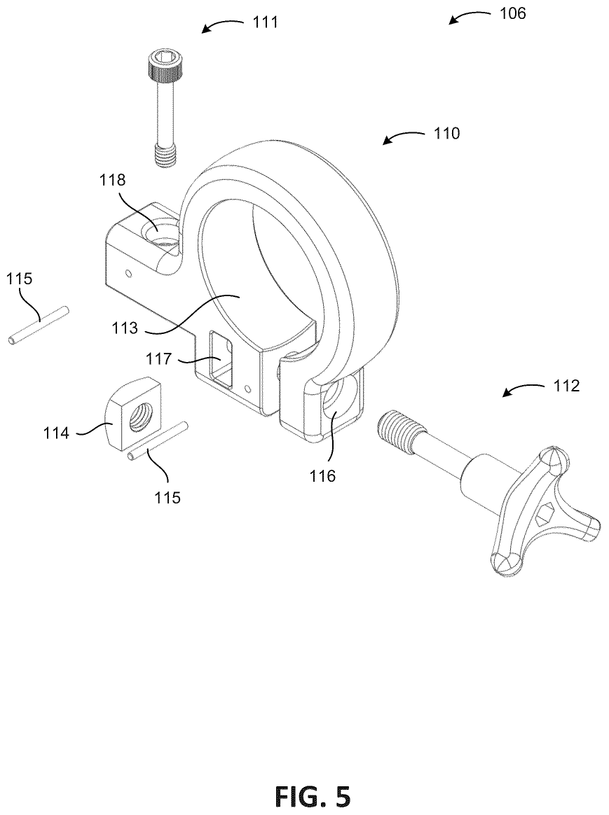

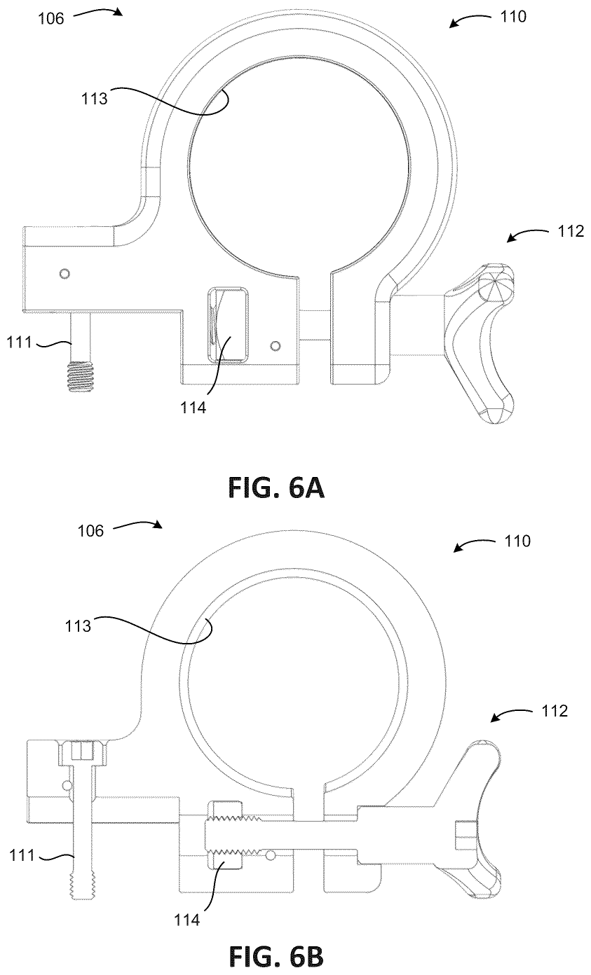

FIGS. 3-6B show various views of strut clamp assemblies 106 that may be utilized to couple the strut assemblies 100 to the pin clamp bodies 300 of the external fixation system 10. Specifically, FIG. 3 is an enlarged perspective view of one end of the external fixation system 10 of FIG. 1, showing two strut clamp assemblies 106. FIG. 4 is an exploded view of the two strut clamp assemblies 106 of FIG. 3, coupled to a pin clamp body 300. FIG. 5 is an exploded, perspective view of one of the strut clamp assemblies 106 of FIG. 4 in isolation. FIG. 6A is a front view of the strut clamp assembly 106 of FIG. 5 after assembly. FIG. 6B is a section view of the strut clamp assembly 106 of FIG. 6A.

Each strut clamp assembly 106 may generally include a clamp 110 having an inner clamp surface 113, a clamp bolt hole 116 formed in the clamp 110 and configured to receive a clamp bolt 112, a clamp nut hole 117 formed in the clamp 110 and configured to receive a clamp nut 114, a clamp fastener hole 118 configured to receive a clamp fastener 111, and one or more clamp pins 115 to retain the clamp bolt 112 and/or the clamp fastener 111 within the clamp 110.

In some embodiments, each strut clamp assembly 106 of the external fixation system 10 may comprise the same/similar parts that may be reproduced to help reduce manufacturing costs. Further, in some embodiments, each strut clamp assembly 106 may be configured to couple with a sphere portion 120 of a strut assembly 100 to form a polyaxial joint 105.

The strut clamp assemblies 106 described above are merely examples of strut attachment assemblies that may be used to secure a bone anchoring body to a strut assembly. In alternative embodiments (not shown), a strut attachment assembly may be attached to a bone anchoring body via other connections such as any mechanical clips, clamps, or other attachment mechanisms known in the art, and may likewise be attached to the strut assembly with other attachment mechanisms.

In some embodiments, the sphere portion 120 may comprise a convex spherical or semi-spherical surface, and the inner clamp surface 113 of the clamp 110 may comprise a concave spherical or semi-spherical surface configured to mate with the sphere portion 120 to form the polyaxial joint 105. The inner clamp surface 113 may fit loosely enough around the sphere portion 120 to allow polyaxial rotation of the sphere portion 120 within the inner clamp surface 113 until the clamp bolt is tightened to cause the inner clamp surface 113 to contract around the sphere portion 120, locking out further polyaxial rotation. In this application, “polyaxial rotation” is rotation about multiple axes. By way of example, the strut clamp assemblies provide relative rotation between the strut assemblies and the pin clamp bodies 300 about three orthogonal axes of rotation. However, in some embodiments, polyaxial rotation may involve rotation about multiple axes but not three orthogonal axes.

Use of two strut clamp assemblies 106 on a pin clamp body 300 may enable multiple strut assemblies 100 to be coupled to the pin clamp body 300, as will be shown and described in more detail below. In some embodiments, the two strut clamp assemblies may be secured at their other ends to another single pin clamp body 300. In such embodiments, the strut assemblies 100 may be generally parallel to and alongside each other, and may help to avoid undesired relative rotation between the pin clamp bodies.

However, in alternative embodiments, the strut assemblies 100 coupled to the pin clamp body 300 may be secured, at their other ends, to different pin clamp bodies 300. In such embodiments, the strut assemblies 100 may extend parallel to each other but in opposite directions, or may be nonparallel to each other. Any number of bones and/or joints may be fixed for healing through the modular use of multiple pin clamp bodies 300 secured to bones and/or bone fragments, and secured together via strut assemblies 100.

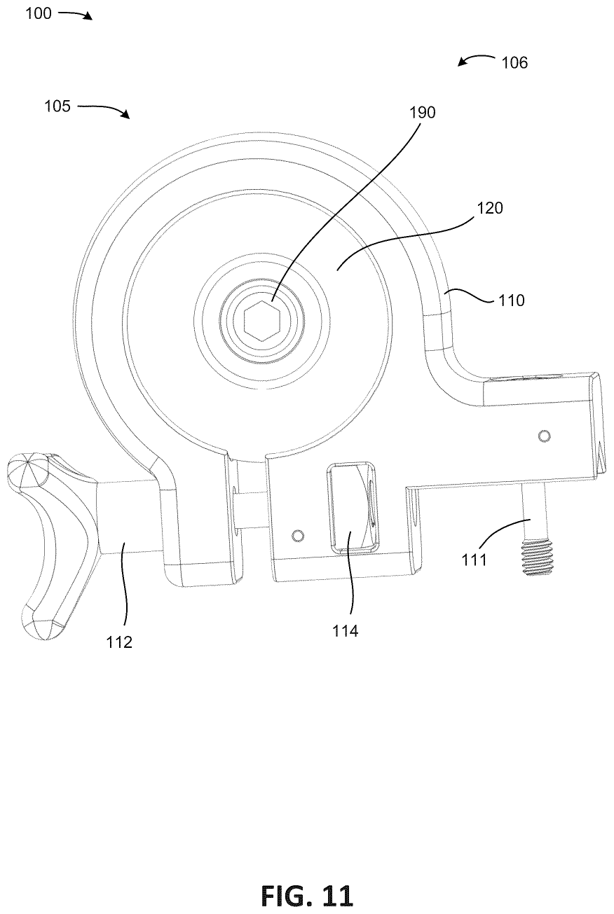

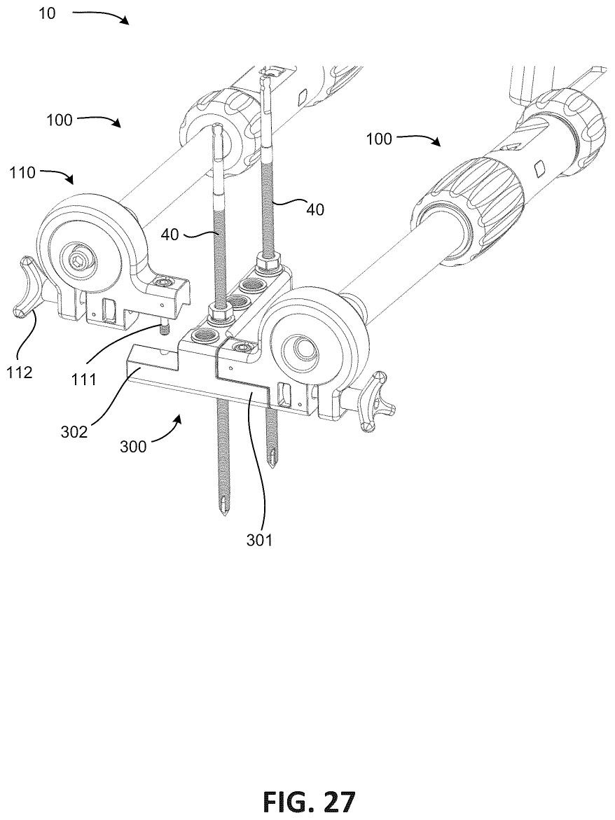

FIG. 11 shows an end view of one of the strut assemblies 100 of the external fixation system 10, with the strut clamp assembly 106 clamped over the sphere portion 120 to form the polyaxial joint 105. FIG. 27 shows how the strut assembly 100 may be removably coupled to a pin clamp body 300 of the external fixation system 10 in a modular fashion. This may allow relatively easy removal (e.g., via disengagement of only a single fastener—the clamp fastener 111) of one of the strut assemblies 100 to gain access to a surgical site and perform a surgical procedure. The utility of such a modular system is also discussed below in more detail with respect to FIGS. 21-30.

FIGS. 3, 4, and 18A-20 show various pin clamp body embodiments of the present disclosure. Each pin clamp body 300 may include a main body portion 305 configured to receive one or more fixation pins 40 through one or more passageways 340 formed in the main body portion 305, a first pin clamp body arm 301, a second pin clamp body arm 302, one or more pin lock inserts 320, and/or one or more pin lock collets 310 receivable within the one or more pin lock inserts 320 to grasp and hold fixation pins 40 placed therethrough. This may allow each fixation pin 40 to be separately tightened to ensure uniform tightening is achieved for each fixation pin 40.

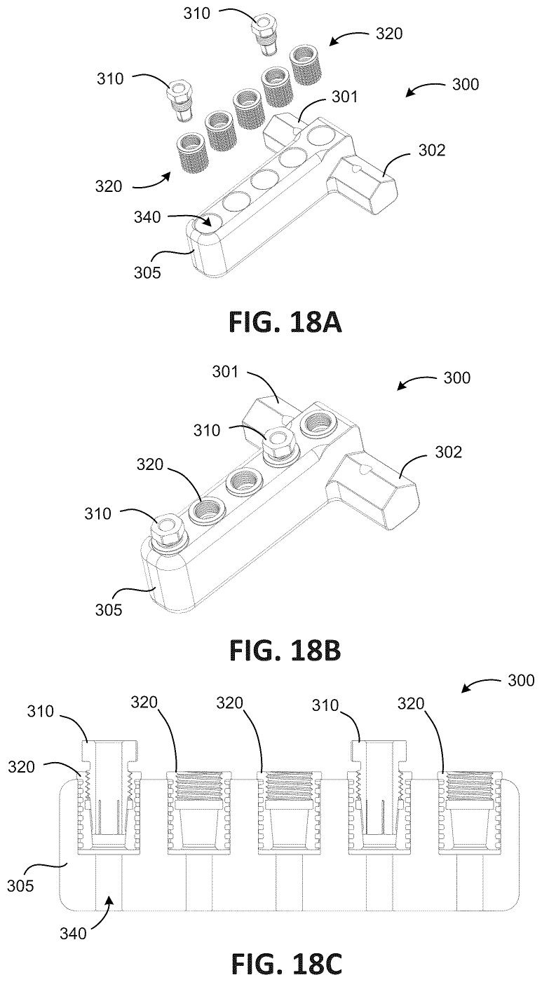

In some embodiments, the pin clamp body 300 may be generally “T-shaped,” as shown in FIGS. 1-4, 12-14, 18A-18C, 21, 22, and 27-30. In alternative embodiments (not shown), the pin clamp body 300 may be generally “L-shaped.” For example, the “T-shaped” pin clamp body shown in FIGS. 18A-18C may be modified to have only one of the first pin clamp body arm 301 and the second pin clamp body arm 302 to form an “L-shaped” pin clamp body. Such a pin clamp body embodiment may be useful for external fixation systems requiring only one strut assembly 100, such as for an external fixation system of an elbow joint (not shown), etc.

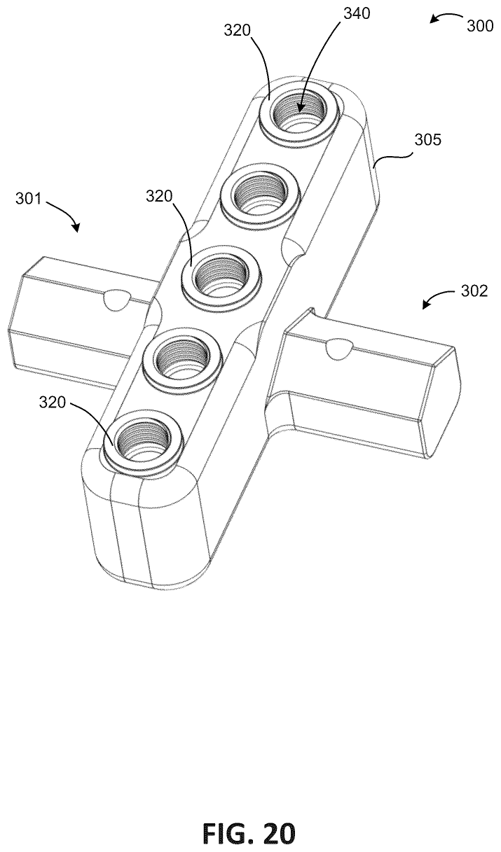

In some embodiments, the pin clamp body 300 may be little/lower case “t-shaped,” as shown in FIG. 20. Further, in some embodiments, the pin clamp body 300 may comprise a partial lower case “t-shaped” pin clamp body (not shown). For example, the lower case “t-shaped” pin clamp body shown in FIG. 20 may be modified to have only one of the first pin clamp body arm 301 or the second pin clamp body arm 302 to form a partial lower case “t-shaped” pin clamp body. This pin clamp body embodiment may be useful for external fixation systems (not shown) requiring only one strut assembly 100, such as for a distal radius external fixation system spanning a wrist joint, etc.

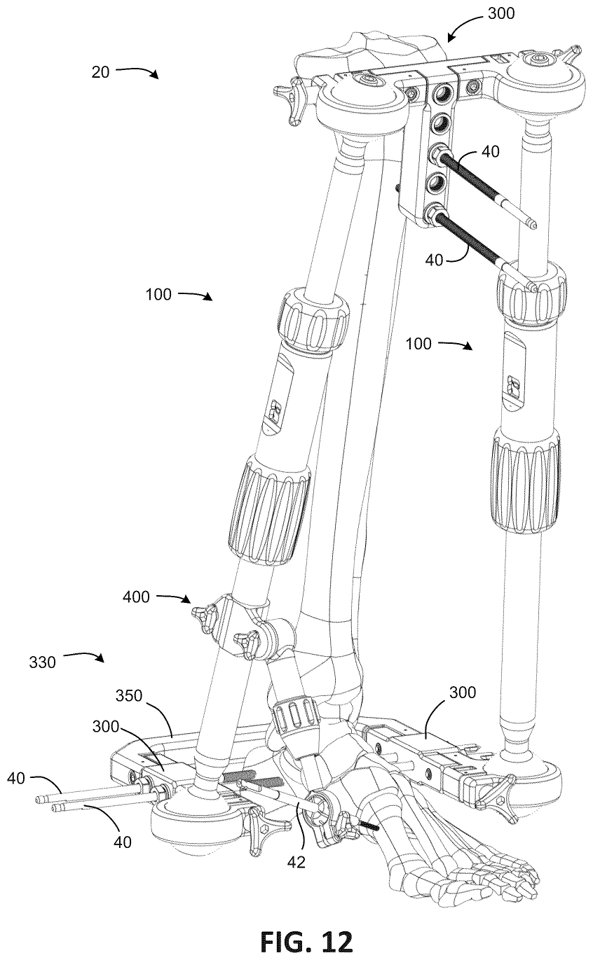

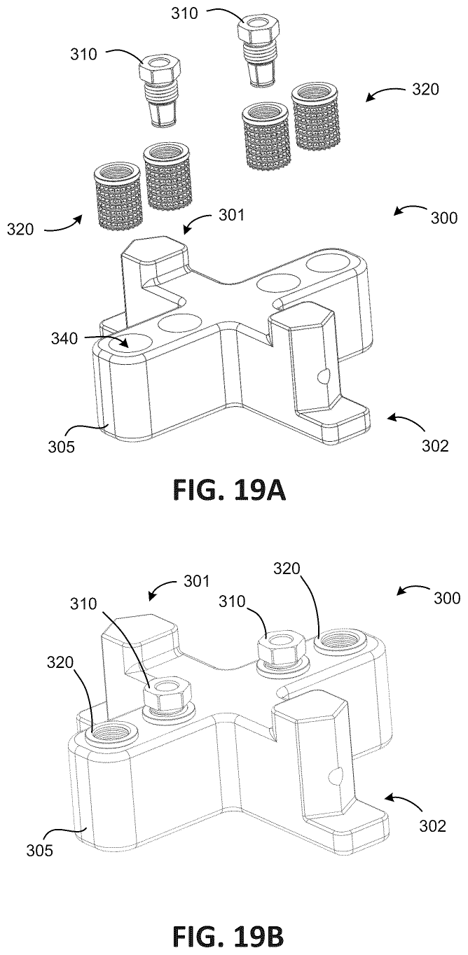

In some embodiments, the pin clamp body 300 may be “X-shaped,” as shown in FIGS. 19A, 19B, and 22-26. Further, in some embodiments, the pin clamp body 300 may be an ankle pin clamp body and/or may be “C-shaped,” such as the pin clamp body 300 shown in FIGS. 12-16 and 22-26.

In some embodiments, the first pin clamp body arm 301 and/or the second pin clamp body arm 302 may have a shape configured to mate with that of the corresponding strut clamp assembly 106 to ensure that the pin clamp body 300 and the strut clamp assembly 106 are properly and consistently located, relative to each other, when attached. For example, the first pin clamp body arm 301 and/or the second pin clamp body arm 302 may have a convex V-shaped surface configured to mate with a corresponding concave V-shaped surface formed in the strut clamp assembly 106. These surfaces may help align/orient the strut clamp assembly 106 with respect to the first pin clamp body arm 301 and/or the second pin clamp body arm 302 during installation, as seen in FIG. 27. However, it will be understood that the first pin clamp body arm 301, the second pin clamp body arm 302, and/or the strut clamp assembly 106 may have any surface shape or surface feature (or no surface shape or surface feature) to help align/orient the strut clamp assembly 106 with respect to the first pin clamp body arm 301 and/or the second pin clamp body arm 302 during installation.

The pin clamp bodies 300 disclosed herein are merely examples of bone anchoring bodies that may be secured to bones and/or bone fragments. In other embodiments (not shown), alternative bone anchoring bodies may be used, and may be anchored to bone in ways aside from the use of pin clamps. For example, a bone anchoring body may be designed to be secured to a bone via screws, unclamped pins, clamps, etc. Bone anchoring bodies according to the present disclosure may be used to secure a first bone in relation to a second bone. The first and second bones may be separate bones, or may be fragments of a single bone that is to be repaired.

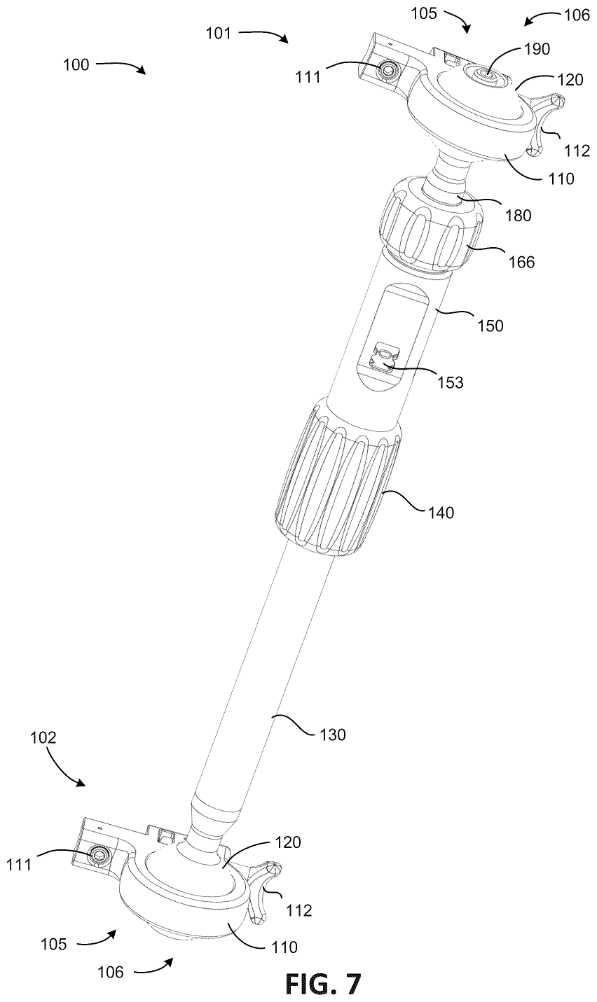

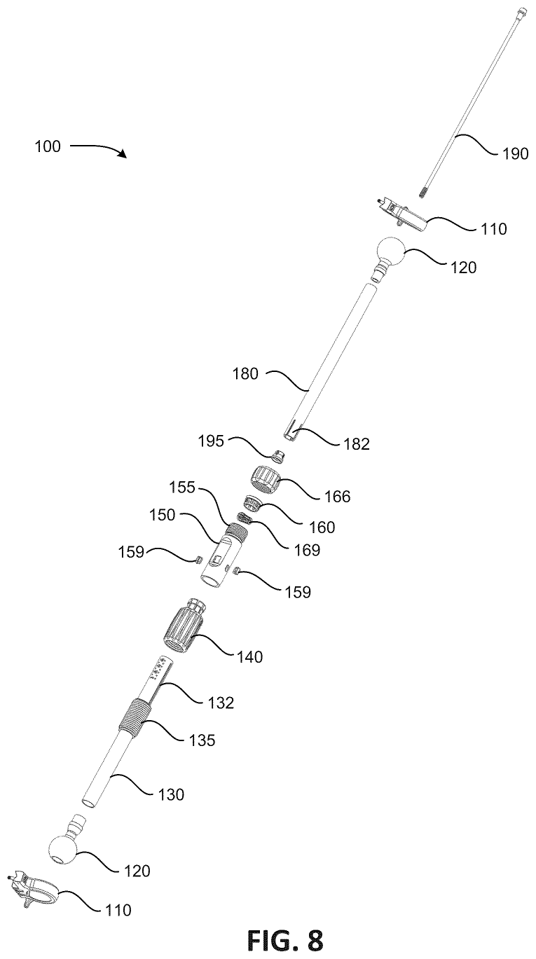

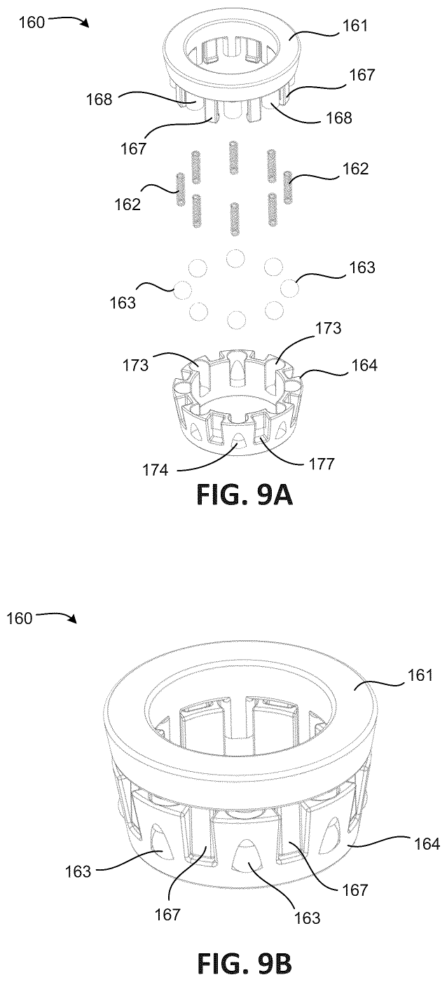

FIGS. 7-10G show various views of a strut assembly 100 that may be utilized with the external fixation systems of the present disclosure. Specifically, FIG. 7 is a perspective view of the strut assembly 100. FIG. 8 is an exploded view of the strut assembly 100. FIG. 9A is an exploded view of a clutch mechanism 160 of the strut assembly 100 shown in FIG. 8. FIG. 9B shows the clutch mechanism 160 of FIG. 9A, after assembly.

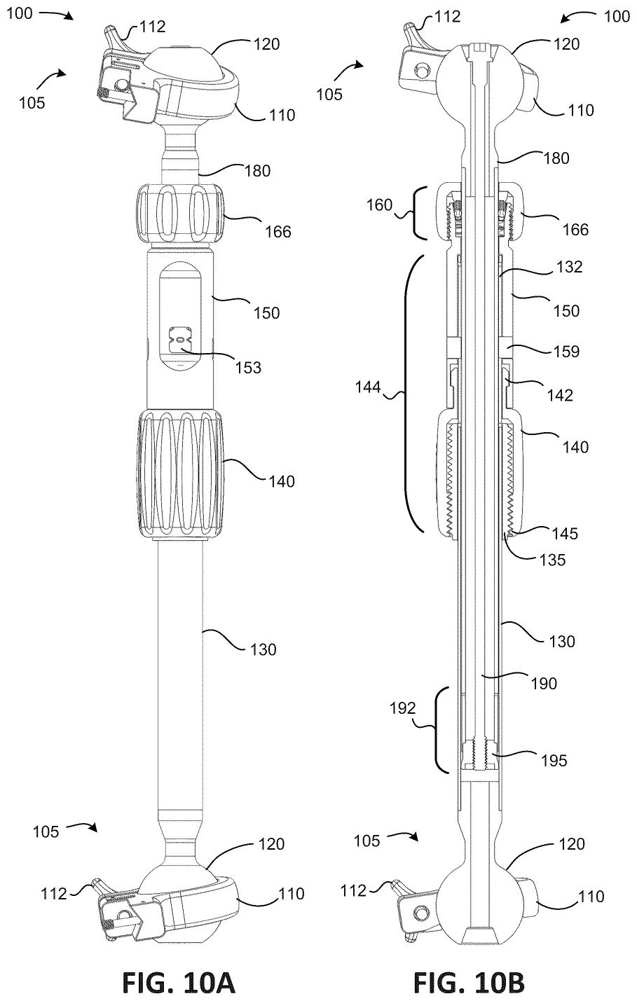

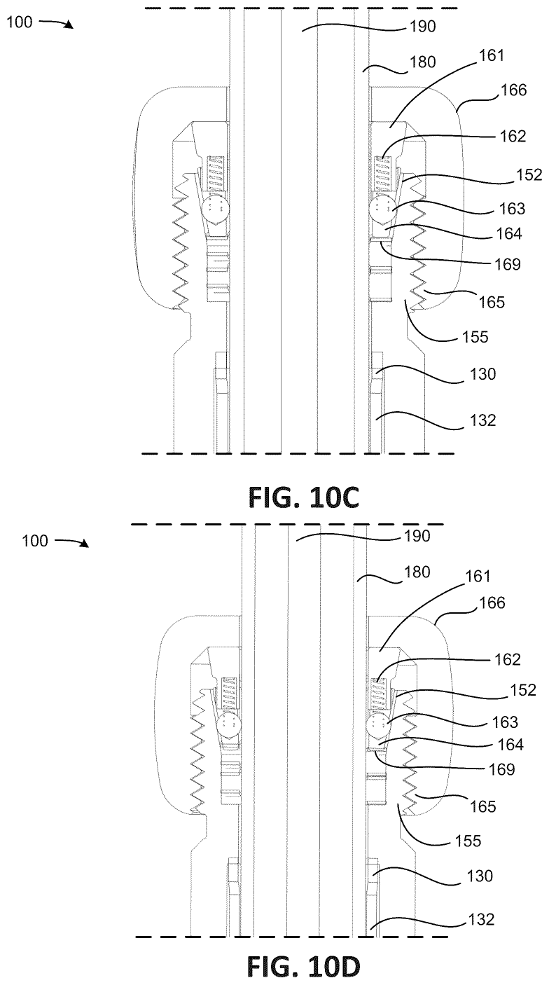

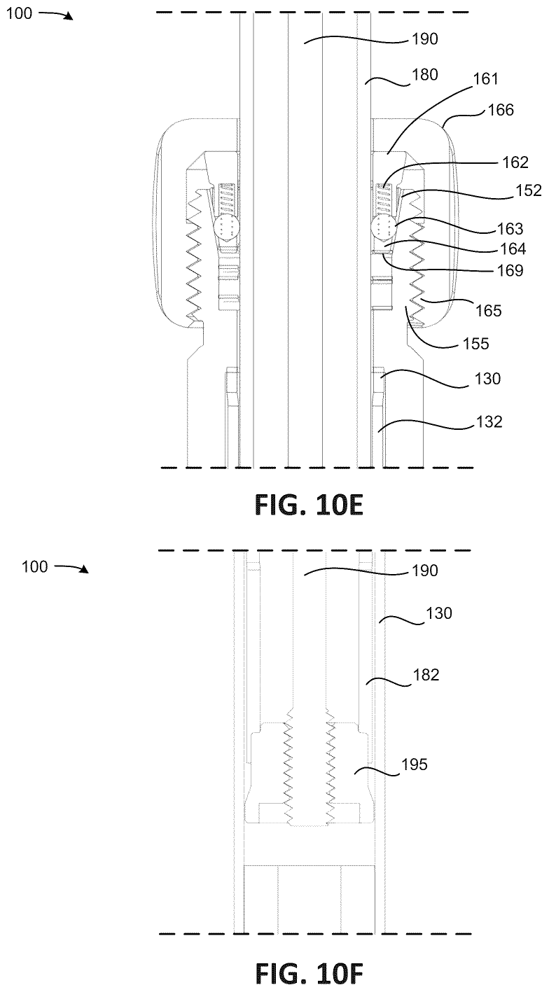

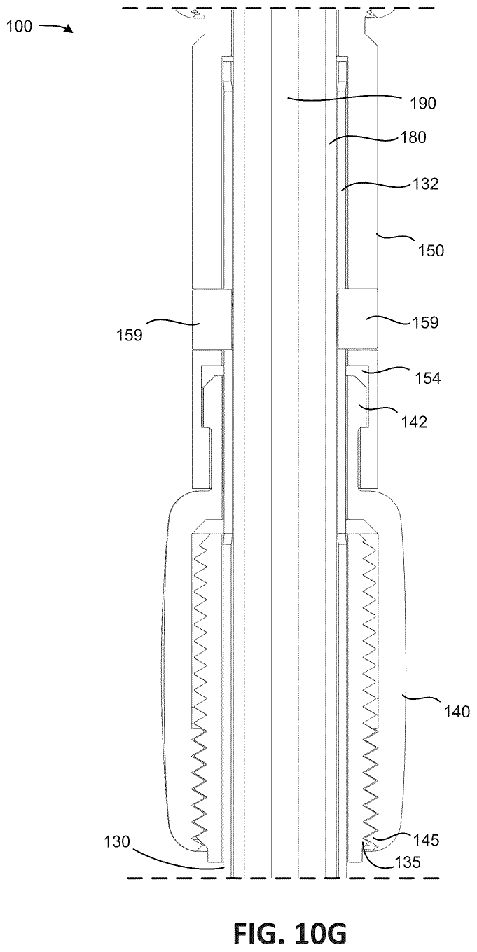

Further, FIG. 10A is a side elevation view of the strut assembly 100. FIG. 10B is a side elevation, section view of the strut assembly 100. FIG. 10C is an enlarged view of the clutch mechanism 160 of the section view of FIG. 10B, in a first configuration. FIG. 10D is an enlarged view of the clutch mechanism 160 of the section view of FIG. 10B, in a second configuration. FIG. 10E is an enlarged view of the clutch mechanism 160 of the section view of FIG. 10B, in a third configuration. FIG. 10F is an enlarged view of a locking mechanism 192 of the section view of FIG. 10B. FIG. 10G is an enlarged view of an adjustment mechanism 144 of the section view of FIG. 10B.

The strut assembly 100 may be coupled to two strut clamp assemblies 106, which may be coupled to two sphere portions 120 located at a proximal end 101 and a distal end 102 of the strut assembly 100, as previously discussed. The strut assembly 100 may also include an outer strut tube 130 having an outer strut tube thread 135 and at least one outer strut tube slot 132 formed in a proximal end of the outer strut tube 130. The strut assembly 100 may also include an adjustment knob 140 having an inner adjustment knob thread 145 and an adjustment knob collet 142 at its proximal end, an adjustment collar 150 having an adjustment window 153, an adjustment collar thread 155, at least one anti-rotation plug 159, an inner annular groove 154, and an inner chamfered surface 152. The strut assembly 100 may also include a resilient member 169 (such as a Lee spring, Belleville washer, etc.), a clutch knob 166 having an inner clutch knob thread 165, an inner strut tube 180 having an inner strut tube collet 182 formed in its distal end, and a locking shaft 190 couplable with an inner strut tube expansion plug 195 to expand the inner strut tube collet 182 and lock the inner strut tube 180 with respect to the outer strut tube 130, as shown in FIG. 10F.

As shown in FIGS. 9A and 9B, the clutch mechanism 160 of the strut assembly 100 may generally include a spring carrier 161, a ball carrier 164, one or more clutch springs 162, and one or more clutch balls 163. The spring carrier 161 may include one or more resilient tabs 167 and one or more spring holders 168 to house the one or more clutch springs 162 therein. The ball carrier 164 may include one or more tab receiver slots 177 to receive the one or more resilient tabs 167 of the spring carrier 161 to assemble the clutch mechanism 160 together, and one or more clutch ball holders 173 to receive the one or more clutch balls 163 therein. The ball carrier 164 may also include one or more clutch ball windows 174 for the one or more clutch balls 163 to at least partially project through the one or more clutch ball windows 174, as shown in FIG. 9B.

Operation of the clutch mechanism 160 will now be described with reference to FIGS. 10C-10D. The clutch knob 166 may be rotated about the proximal end of the adjustment collar 150 via engagement of the inner clutch knob thread 165 with the adjustment collar thread 155.

When the clutch knob 166 is rotated to a first position (e.g., more proximally, or toward the top in FIG. 10C) with respect to the adjustment collar 150, the resilient member 169 may push the clutch mechanism 160 proximally to disengage the one or more clutch balls 163 from engaging the surfaces of the inner strut tube 180 and the inner chamfered surface 152 of the adjustment collar 150. This may allow the inner strut tube 180 to freely translate distally and proximally with respect to (e.g., within) the outer strut tube 130.

This first position may be useful when an operator desires to make large-scale adjustments to lengthen or shorten a strut assembly 100. Optionally, the surgeon may do this prior to coupling the strut assembly 100 to one or more pin clamp bodies 300. This first position may be known as an “unlocked position.”

When the clutch knob 166 is rotated to a second position (e.g., slightly distal to the first position, or slightly lower in FIG. 10D) with respect to the adjustment collar 150, the clutch mechanism 160 may start pushing against the resilient member 169 and the one or more clutch springs 162 may start pushing the more clutch balls 163 downward, forcing them to protrude out of the one or more clutch ball windows 174. This may allow some degree of engagement for the one or more clutch balls 163 with the surfaces of the inner strut tube 180 and the inner chamfered surface 152 of the adjustment collar 150, which may allow the inner strut tube 180 to freely translate distally with respect to the outer strut tube 130 but resist proximal translation of the inner strut tube 180 with respect to the outer strut tube 130 as the clutch balls 163 are pushed toward the inner chamfered surface 152 by significant motion of the inner strut tube 180 further into the outer strut tube 130.

This second position may be useful when an operator desires to fine tune a length of the strut assembly 100 to control a distance between a first pin clamp body 300 and a second pin clamp body 300 that may be spanned by the strut assembly 100. The strut assembly 100 may be freely lengthened, but may allow shortening with some resistance that can be overcome by the surgeon. This position may be known as a “restricted position.”

When the clutch knob 166 is rotated to a third position (e.g., distal to the second position, or yet lower than is shown in FIG. 10D—for example, between the positions of FIGS. 10D and 10E) with respect to the adjustment collar 150, the clutch mechanism 160 may push harder against the resilient member 169 and the one or more clutch springs 162 may push the more clutch balls 163 downward with greater force, forcing them to protrude out of the one or more clutch ball windows 174 with more force. This may engage the one or more clutch balls 163 with the surfaces of the inner strut tube 180 and the inner chamfered surface 152 of the adjustment collar 150 with greater force, which may allow the inner strut tube 180 to translate distally with respect to the outer strut tube 130 but substantially prevent proximal translation of the inner strut tube 180 with respect to the outer strut tube 130 as the clutch balls 163 are pushed toward the inner chamfered surface 152 by virtually any motion of the inner strut tube 180 further into the outer strut tube 130.

This third position may be useful when an operator desires to increase a length of the strut assembly 100 and avoid a possible decrease in the length of the strut assembly 100 (e.g., when the surgeon is trying to stretch a joint/fracture to is proper length against the natural tension in the muscles, and the surgeon does not want any decrease in the length of the strut assembly 100 during/after this procedure). This position may be known as a “partially-locked position.”

When the clutch knob 166 is rotated to a fourth position (e.g., more distal of the third position, or further downward in FIG. 10E) with respect to the adjustment collar 150, the clutch mechanism 160 may push even harder against the resilient member 169 and the one or more clutch springs 162 (and/or the one or more spring holders of the spring carrier 161) may push the more clutch balls 163 downward with even greater force, forcing them to protrude out of the one or more clutch ball windows 174 with increased force. This will engage the one or more clutch balls 163 with the surfaces of the inner strut tube 180 and the inner chamfered surface 152 of the adjustment collar 150 with even more force, which may prevent the inner strut tube 180 from translating distally and/or proximally with respect to the outer strut tube 130, as the clutch balls 163 are in constant contact with the inner chamfered surface 152, exerting pressure against the inner strut tube regardless of motion of the inner strut tube 180 into or out of the outer strut tube 130.

This fourth position may be useful when an operator desires to maintain a selected length of the strut assembly 100. The strut assembly 100 may be resistant to both shortening and elongation in this configuration. This position may be known as a “locked position.”

Advantageously, the clutch mechanism 160 may be easily moved by the surgeon between the first, second, third, and/or fourth positions. In some embodiments, only limited rotation of the clutch knob 166 may be needed to move between the locked and unlocked positions. For example, in one embodiment, the clutch knob 166 may only need to be rotated ¼ turn to move between the locked and unlocked positions. This may ease adjustment and/or locking of the length of the strut assembly 100 by the surgeon, and may serve to minimize patient discomfort during the adjustment and/or locking processes.

The inner strut tube 180 may be further locked in place with respect to the outer strut tube 130 by rotating the locking shaft 190 to draw the inner strut tube expansion plug 195 proximally to expand the inner strut tube collet 182 and lock the inner strut tube 180 in place with respect to the outer strut tube 130, as shown in FIG. 10F. The inner strut tube 180 may have a drive feature, such as the hex socket shown in FIG. 11, that facilitates rotation of the inner strut tube 180 via a tool such as a hex wrench. In the locked position (e.g., with the inner strut tube collet 182 expanded to engage the interior of the outer strut tube 130 as in FIG. 10F), the inner strut tube expansion plug 195 and the inner strut tube collet 182 may define a locking mechanism that prevents shortening and lengthening of the strut assembly 100, in addition to the locking provided by the clutch mechanism 160.

Referring to FIG. 10G, a length of the outer strut tube 130 projecting from the adjustment knob 140 may be fine-tuned via the adjustment mechanism 144, as will now be described in more detail.

The adjustment knob collet 142 may reside in the inner annular groove 154 formed in the adjustment collar 150 to couple the adjustment knob 140 with the adjustment collar 150, while allowing the adjustment knob 140 to freely rotate with respect to the adjustment collar 150. The inner adjustment knob thread 145 of the adjustment knob 140 may engage the outer strut tube thread 135, such that rotation of the adjustment knob 140 will cause the outer strut tube 130 to translate proximally and/or distally with respect to the adjustment knob 140 as the adjustment knob 140 is rotated in either direction. The at least one anti-rotation plug 159 coupled to the adjustment collar 150 may also be received within the at least one outer strut tube slot 132 to prevent rotation of the outer strut tube 130 as is translates proximally and/or distally with respect to the adjustment knob 140 and/or the adjustment collar 150. In this manner, an overall length of the strut assembly 100 may be adjusted by rotating the adjustment knob 140. The amount of adjustment to the length of the strut assembly 100 may be viewed through the adjustment window 153 as the outer strut tube 130 translates proximally and/or distally with respect to the adjustment collar 150 (e.g., see FIGS. 7 and 10A).

The adjustment mechanism 144 may provide fine tuning of the length of the strut assembly 100. In some embodiments, the adjustment mechanism 144 may provide an adjustment range of about 60 mm. Advantageously, the mechanical advantage provided by the adjustment mechanism 144 may be sufficient that adjustments made via the adjustment mechanism 144 need not be locked in place with any supplemental locking mechanism.

In some embodiments, the adjustment mechanism 144 may provide an adjustment range of less than 60 mm such as, for example, 5 mm, 10 mm, 20 mm, 30 mm, 40 mm, or 50 mm. In some embodiments, the adjustment mechanism 144 may provide an adjustment range of greater than 60 mm such as, for example, 70 mm, 80 mm, 90 mm, 100 mm, or 120 mm.

In some embodiments, the adjustment mechanism 144 may initially be provided in a fully-extended configuration (i.e., at maximum length), and may thus provide its entire adjustment range (for example, 60 mm) for extra retraction. In alternative embodiments, the adjustment mechanism 144 may initially be provided in a fully-retracted configuration (i.e., at minimum length), and may thus provide its entire adjustment range (for example, 60 mm) for extra extension (e.g., lengthening). In yet other embodiments, the adjustment mechanism may initially be provided in a centered configuration (i.e., midway between minimum and maximum lengths), and may thus divide its adjustment range evenly between retraction and extension.

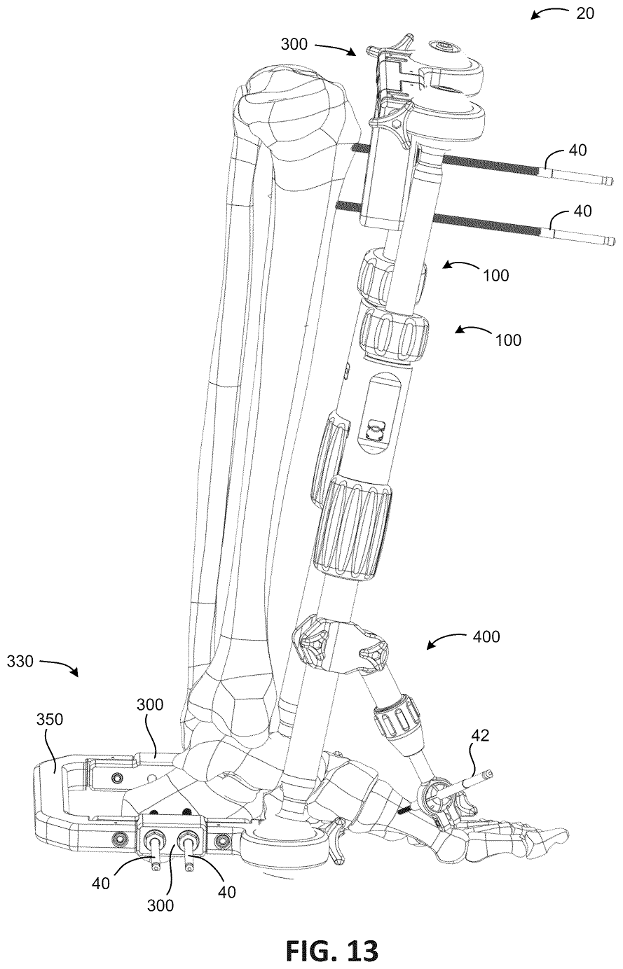

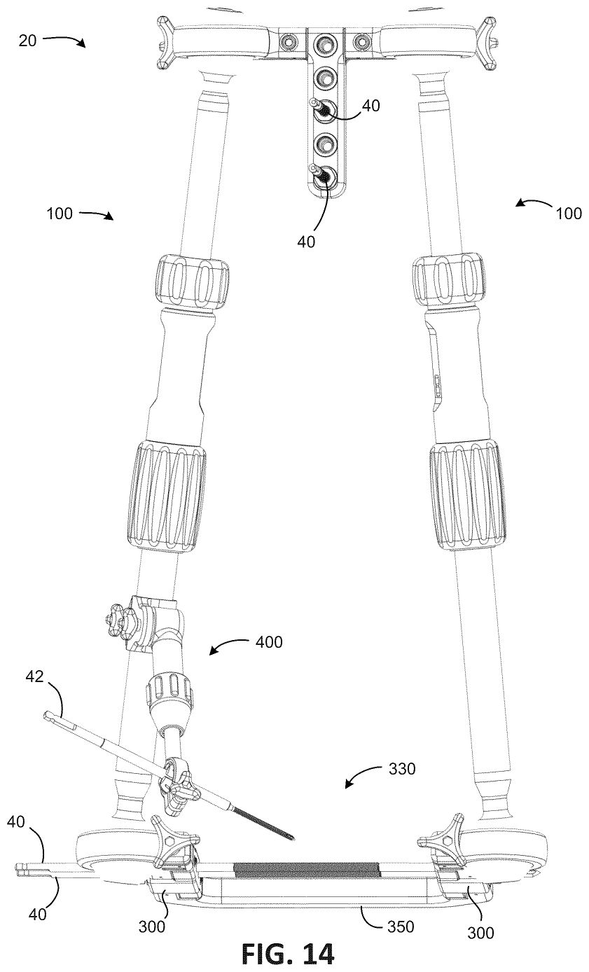

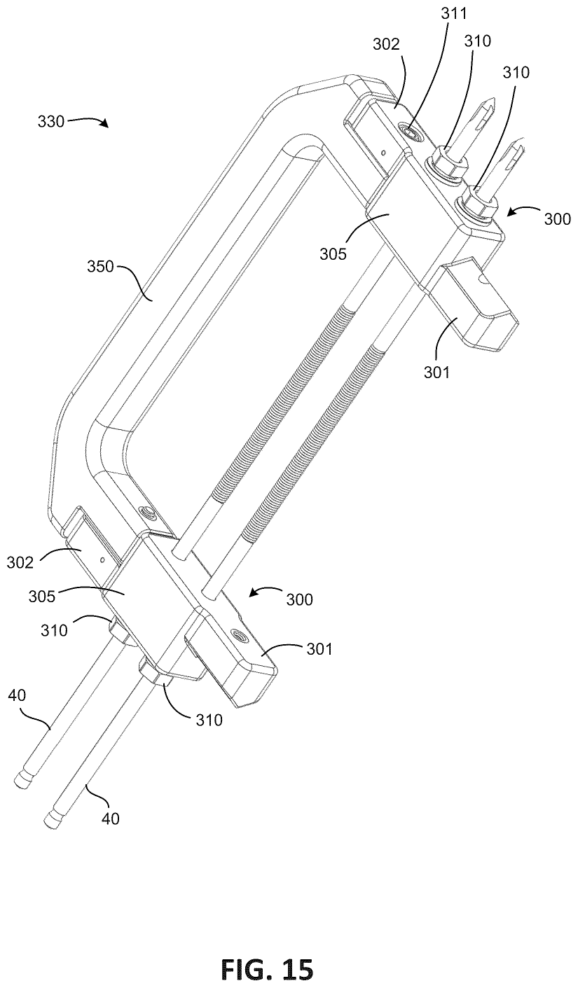

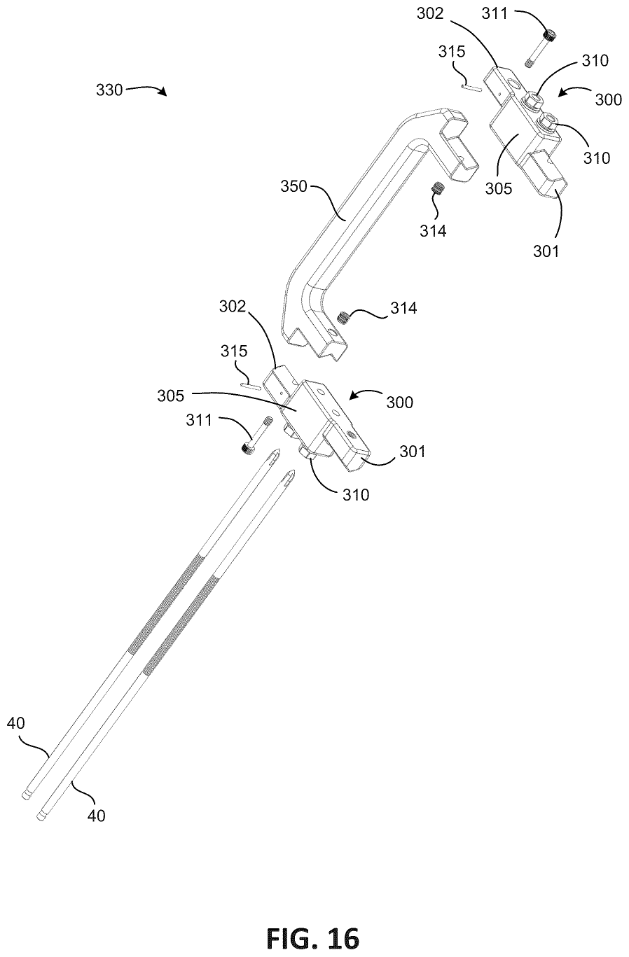

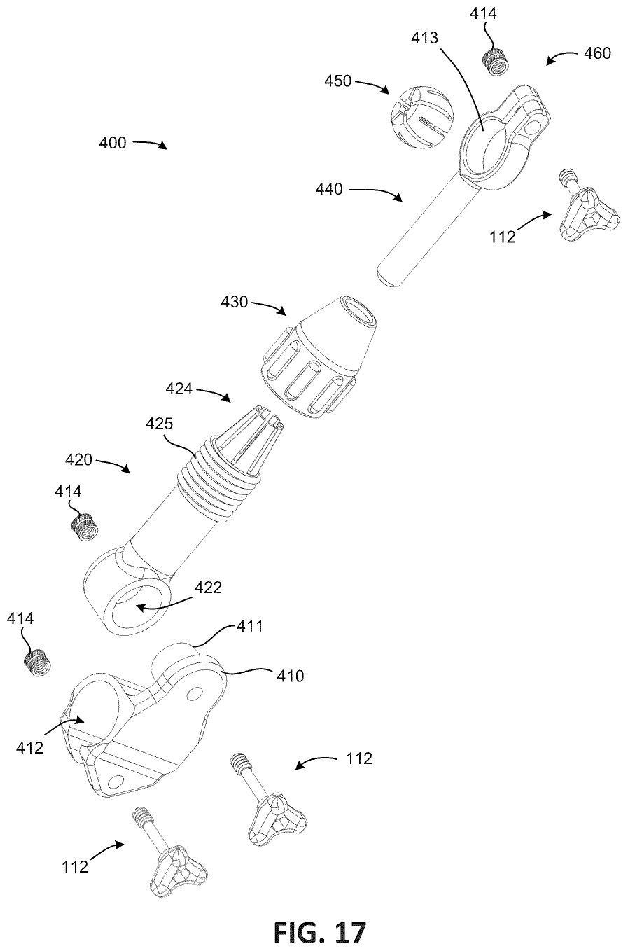

FIGS. 12-17 provide various views of an external fixation system 20 configured to span an ankle joint, according to an embodiment of the present disclosure. Specifically, FIG. 12 is a perspective view of the external fixation system 20, secured to the bones of an ankle. FIG. 13 is an alternative perspective view of the external fixation system 20, secured to the ankle. FIG. 14 is another alternative perspective front view of the external fixation system 20 in isolation. FIG. 15 is a perspective view of an ankle pin clamp assembly 330 of the external fixation system 20. FIG. 16 is an exploded view of the ankle pin clamp assembly 330 of FIG. 15. FIG. 17 is an exploded view of an auxiliary pin clamp assembly 400 of the external fixation system 20.

In some embodiments, the external fixation system 20 may include a first strut assembly 100, a second strut assembly 100, a pin clamp body 300 coupled to a tibia of the patient, an ankle pin clamp assembly 330 coupled to an ankle bone (e.g., a calcaneal bone) of the patient, one or more fixation pins 40, and an auxiliary pin clamp assembly 400 coupled to one of the strut assemblies 100 with a metatarsal fixation pin 42 fixed to a bone of the foot (e.g., a metatarsal bone, etc.).

As shown in FIGS. 15 and 16, the ankle pin clamp assembly 330 may include two pin clamp bodies 300 or two ankle pin clamp bodies 300, an ankle bridge 350 spanning the two ankle pin clamp bodies 300, two fixation pins 40, four pin lock collets 310, two clamp body fasteners 311, two clamp body nuts 314, and two clamp body pins 315.

As shown in FIG. 17, the auxiliary pin clamp assembly 400 may generally include an auxiliary base that can be adjustably secured along the length of the first strut assembly 100 or the second strut assembly 100. In FIG. 17, the base may take the form of a clamp base 410 having a base opening 412 and a base arm 411, clamp bolts 112, clamp nuts 414, a clamp tube 420 having a tube opening 422 to receive the base arm 411 therein, tube threading 425, and a tube collet 424, a clamp knob 430, a clamp rod 440 having a distal clamp 460 with an inner distal clamp surface 413, and a clamp sphere 450.

The clamp base 410 may be attached to a strut assembly 100 by placing the base opening 412 around the outer strut tube 130 of the strut assembly 100 (e.g., see FIGS. 12-14). The clamp base 410 may be translated along the length of the strut assembly 100 to a desired location. The desired location for the clamp base 410 may then be maintained by inserting the clamp bolt 112 into the clamp base 410 and tightening the clamp bolt 112.

The clamp tube 420 may be selectively rotated about the base arm 411 to any desired degree of rotation by loosening the clamp bolt 112 that is placed through the base arm 411, rotating the clamp tube 420 to a desired degree of rotation, then tightening the clamp bolt 112 again to maintain this position. The base arm 411 may optionally have an expandable structure such as a collet that expands in response to axial compression provided by tightening the corresponding clamp bolt 112. The expandable structure may expand to engage the interior surface of the tube opening to restrict or prevent relative rotation between the clamp tube 420 and the clamp base 410.

The clamp rod 440 may be inserted through the clamp knob 430 and into the clamp tube 420. The tube collet 424 and the clamp knob 430 may have conical shapes, such that the clamp knob 430 may be rotated to compress the tube collet 424 against the clamp rod 440 to capture/hold the clamp rod 440 in a desired translational and/or rotational position. In this manner, a length of the clamp rod 440 protruding from the clamp knob 430 and/or a rotational position of the clamp rod 440 with respect to the clamp tube 420 may be selected by the operator and then locked into position.

The clamp sphere 450 may act as an auxiliary pin guide by enabling a pin to be secured to bone at a polyaxially adjustable angle relative to the auxiliary base. The clamp sphere 450 may be received within the distal clamp 460 that is coupled with the clamp rod 440. The clamp sphere 450 may have an at least partially spherical convex outer surface, and the distal clamp 460 may have an inner distal clamp surface 413 with an at least partially spherical concave surface to form a polyaxial connection with the clamp sphere 450. The clamp sphere 450 may have one or more slits and a central aperture formed therein and configured to receive the metatarsal fixation pin 42 therethrough. A polyaxial orientation of the clamp sphere 450 may be selected by the operator, and the clamp bolt 112 may be placed through the distal clamp 460 and tightened to maintain the selected polyaxial orientation and/or to grasp/hold a metatarsal fixation pin 42 that is placed through the central aperture formed in the clamp sphere 450, as shown in FIGS. 12-14.

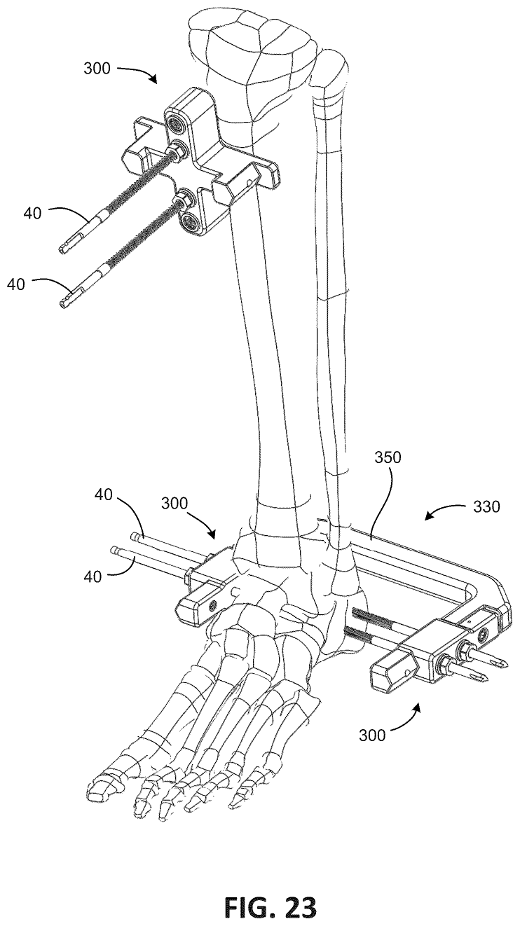

FIGS. 23-26 show an exemplary method for assembling the external fixation system 20 of FIGS. 12-17 across an ankle joint of a patient. The method of FIGS. 23-26 is merely one of many methods in which the external fixation system 20 may be employed. Further, the method of FIGS. 23-26 may be utilized with different hardware than that specifically shown in FIGS. 12-17.

In a first step, a pin clamp body 300 may be pinned to a proximal tibia of the patient, as shown in FIG. 23. In some embodiments, the pin clamp body 300 may be an “X-shaped” pin clamp body, as shown in FIGS. 19A, 19B, and 22-26. However, it will be understood that the pin clamp body 300 may have any pin clamp body style or shape that is disclosed or contemplated herein, or any that would be envisioned by a person of skill in the art with the aid of the present disclosure.

In a second step of the procedure, the ankle pin clamp assembly 330 shown in FIGS. 12-16 may be assembled and/or pinned through a calcaneal bone of the patient, as previously described herein.

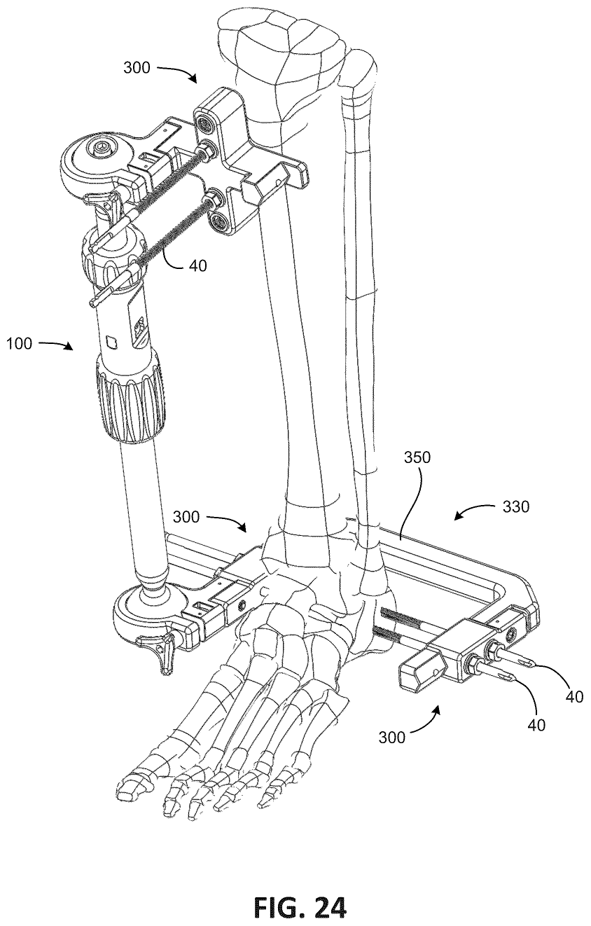

In a third step of the procedure, a first strut assembly 100 may be placed intermediate each of the pin clamp bodies 300 and coupled thereto, as shown in FIG. 24. A process for coupling a strut assembly 100 to a pin clamp body 300 has been generally described herein with respect to FIG. 27. The length of the first strut assembly 100 may be adjusted, as previously discussed herein, to selectively control a distance between the pin clamp bodies 300 along the first strut assembly. The length of the first strut assembly 100 may be locked as previously described.

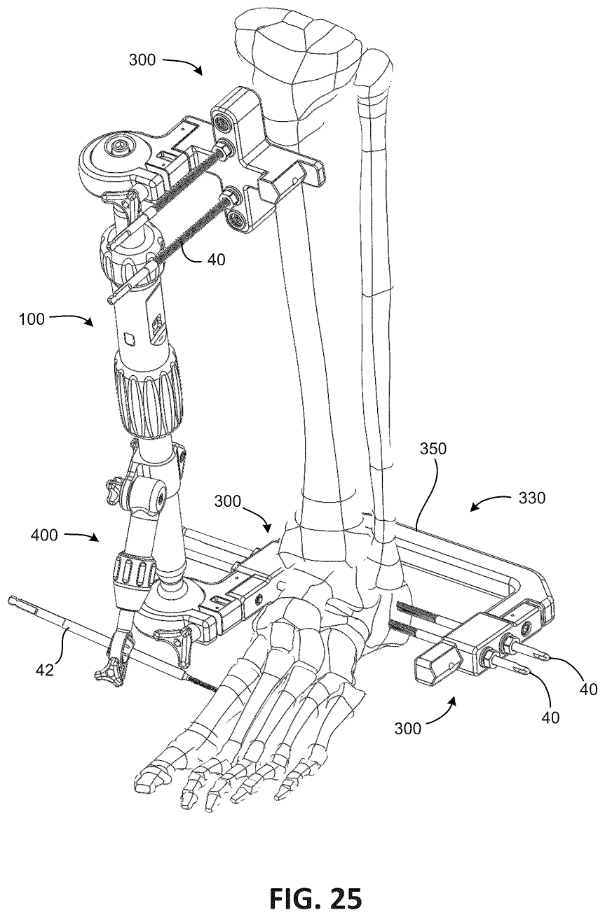

In a fourth step of the procedure, the auxiliary pin clamp assembly 400 may be coupled to the first strut assembly 100 (see FIG. 25) and oriented as desired by the surgeon, as previously discussed herein. A metatarsal fixation pin 42 may be fixed to a bone of the patient's foot at a desired location and held in position by the auxiliary pin clamp assembly 400 to achieve greater stability for the external fixation system. The auxiliary pin clamp assembly 400 may be locked in place, as also previously described.

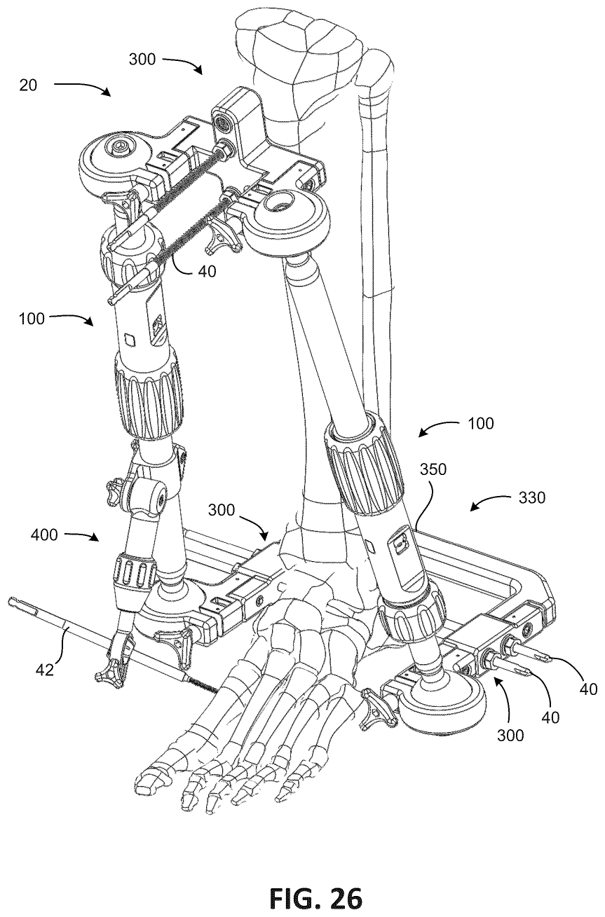

In a fifth step of the procedure, a second strut assembly 100 may be placed intermediate the pin clamp bodies 300 on the calcaneus and the tibia, and coupled thereto, as shown in FIG. 26. The length of the second strut assembly 100 may be adjusted, as previously discussed herein, to selectively control a distance between the pin clamp bodies 300 along the second strut assembly. The length of the second strut assembly 100 may be locked as previously described.

Optionally, or in addition thereto, the operator may then make any additional fine-tuning adjustments to the lengths of either strut assembly, as desired. If desired, the locking of length and/or configuration of the first strut assembly 100, the second strut assembly 100, and/or the auxiliary pin clamp assembly 400 may be delayed until are all in the desired positions. Once all have been placed and adjusted as desired, the surgeon may lock the length and/or configuration of each of the first strut assembly 100, the second strut assembly 100, and/or the auxiliary pin clamp assembly 400.

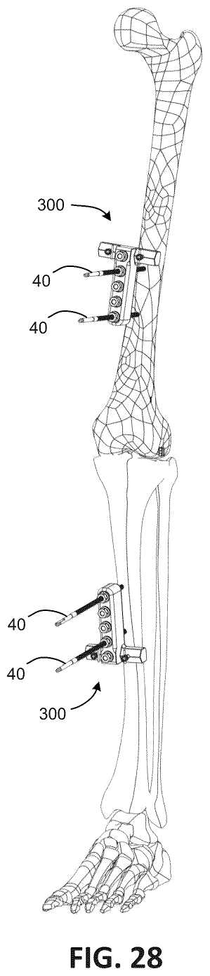

FIG. 21 shows the external fixation system 10 of FIGS. 1-3 spanning a knee joint of a patient, and FIGS. 28-30 show an exemplary method for installing the external fixation system 10 across the knee joint of the patient. The method of FIGS. 28-30 is merely one of many methods in which the external fixation system 10 may be employed. Further, the method of FIGS. 28-30 may be utilized with different hardware than that specifically shown in FIGS. 1-3.

In a first step of the procedure, a pin clamp body 300 may be pinned to a femur of the patient, and another pin clamp body 300 may be pinned to a tibia of the patient, as shown in FIG. 28. In some embodiments, the pin clamp bodies 300 may be “T-shaped” pin clamp bodies, as shown in FIGS. 1-4, 12-14, 18A-18C, 21, 22, and 27-30. However, it will be understood that the pin clamp bodies 300 may have any pin clamp body style or shape that is disclosed or contemplated herein, or would be envisioned by a person skilled in the art with the aid of the present disclosure.

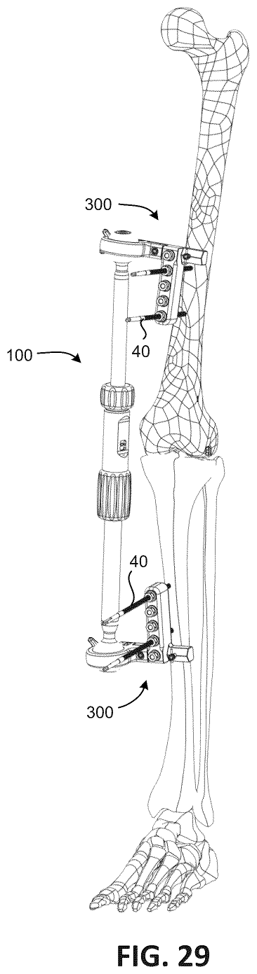

In a second step of the procedure, a first strut assembly 100 may be placed intermediate each of the pin clamp bodies 300 and coupled thereto, as shown in FIG. 29 (see also FIG. 27). The length of the first strut assembly 100 may be adjusted, as previously discussed herein, to selectively control a distance between the pin clamp bodies 300 along the first strut assembly 100. The length of the first strut assembly 100 may be locked, as described above.

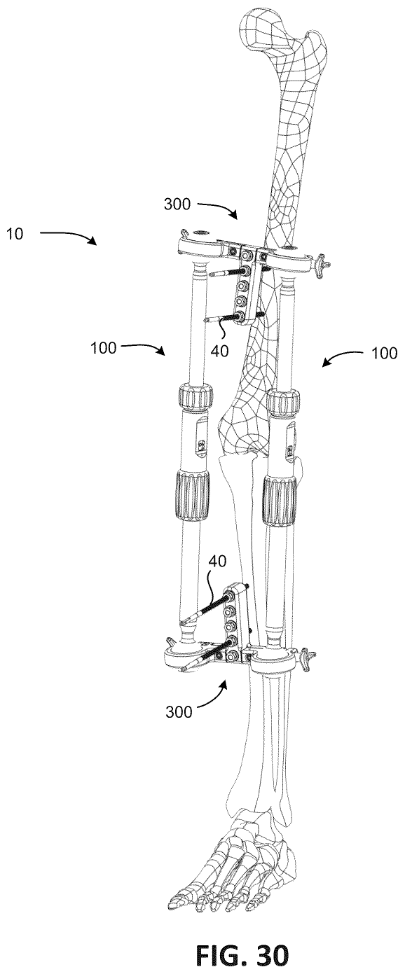

In a third step of the procedure, a second strut assembly 100 may be placed intermediate each of the pin clamp bodies 300 and coupled thereto, as shown in FIG. 30. The length of the second strut assembly 100 may be adjusted, as previously discussed herein, to selectively control a distance between the pin clamp bodies 300 along the second strut assembly 100. The length of the second strut assembly 100 may be locked, as described above.

Optionally, or in addition thereto, the operator may then make any additional fine-tuning adjustments to the lengths of either strut assembly, as desired. If desired, the locking of length of the first strut assembly 100 and/or the second strut assembly 100 may be delayed until are all in the desired positions. Once all have been placed and adjusted as desired, the surgeon may lock the length of the first strut assembly 100 and/or the second strut assembly 100.

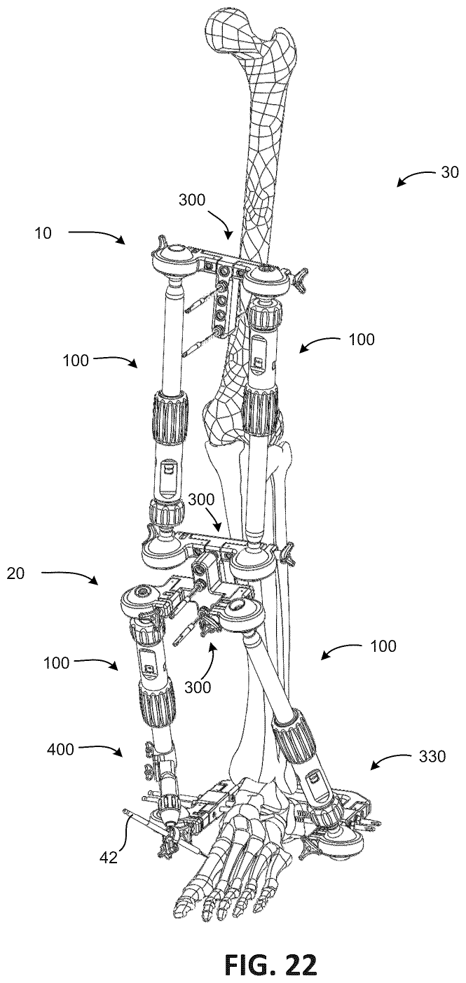

FIG. 22 shows a two-level external fixation system 30 spanning a knee joint and an ankle joint, according to an embodiment of the present disclosure. The two-level external fixation system may comprise components of the external fixation systems 10 and 20 of FIGS. 21 and 12 coupled with each other, as shown in FIG. 22. Moreover, a procedure for assembling the two-level external fixation system 30 may include some or all of the procedure steps previously described with respect to FIGS. 23-30. These steps may be carried out in various orders—for example, the external fixation system 10 may be placed and locked first, followed by the external fixation system 20. In the alternative, the order may be reversed. As yet another alternative, the external fixation system 10 and the external fixation system 20 may both be placed (in either order) and adjusted as desired, and then both the external fixation system 10 and the external fixation system 20 may be locked as described above.

For the various methods described herein, the method steps and/or actions may be interchanged with one another. In other words, unless a specific order of steps or actions is required for proper operation of the embodiment, the order and/or use of specific steps and/or actions may be modified. Further, in some embodiments, method steps set forth above may be omitted, replaced with alternatives, and/or supplemented with additional steps not specifically described herein.

The technology described herein may utilize one or more pin clamp bodies that provide at least one polyaxial joint to enable a highly adaptable connection between two bones or bone fragments via at least one strut assembly. The devices, systems, kits, and methods of the present disclosure can provide external fixation systems which may be disposable and/or low cost. Each of the external fixation systems disclosed herein may be provided unassembled, pre-assembled, or partially assembled in one or more kits (not shown) which may also include tools, fixation members such as pins, etc.

Various materials may be used to form the components of the external fixation system 10, the external fixation system 20, and/or the two-level external fixation system 30. In some examples, many of the components may be formed of a sturdy yet substantially radiolucent material such as aluminum in order to enhance visualization of the patient's bone structure via X-ray. Where needed, some components may be formed of higher strength materials such as steel and titanium. For example, the strut assemblies 100 may be generally formed of aluminum, except that the various load-bearing surfaces of the clutch mechanism 160, the adjustment mechanism 144, and/or the locking mechanism defined by the inner strut tube expansion plug 195 and the inner strut tube collet 182 may be formed of titanium and/or stainless steel. In some embodiments, lighter weight materials, such as plastics, may be used for some components of the external fixation system 10, the external fixation system 20, and/or the two-level external fixation system 30.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

What is claimed is:

1. An external fixation system for securing a first bone of a patient relative to a second bone of the patient, the external fixation system comprising:
a first bone anchoring body configured to be secured to the first bone;
a second bone anchoring body configured to be secured to the second bone; and
a first strut assembly comprising:
a first end configured to be removably coupled to the first bone anchoring body;
a second end configured to be removably coupled to the second bone anchoring body;
an intermediate portion that extends between the first end and the second end, the intermediate portion having a length that is adjustable; and
a clutch mechanism that is movable between:
an unlocked position in which the clutch mechanism allows both increase and reduction in the length; and
a restricted position in which the clutch mechanism allows one of increase and reduction in the length while restricting the other of increase and reduction in the length.

2. The external fixation system of claim 1, further comprising:
a first strut attachment assembly that is attachable to the first bone anchoring body and to the first end of the first strut assembly; and
a second strut attachment assembly that is attachable to the second bone anchoring body and to the second end of the first strut assembly.

3. The external fixation system of claim 2, wherein:
the first end of the first strut assembly comprises a first sphere portion;
the second end of the first strut assembly comprises a second sphere portion;
the first bone anchoring body comprises a first arm;
the second bone anchoring body comprises a second arm;
the first strut attachment assembly comprises:
a first fastener configured to secure the first strut attachment assembly to the first arm; and
a first strut clamp assembly comprising a first clamp comprising a first inner clamp surface configured to receive and be tightened around the first sphere portion; and
the second strut attachment assembly comprises:
a second fastener configured to secure the second strut attachment assembly to the second arm; and
a second strut clamp assembly comprising a second clamp comprising a second inner clamp surface configured to receive and be tightened around the second sphere portion.

4. The external fixation system of claim 3, wherein:
the first strut clamp assembly is securable to the first arm by actuating only the first fastener; and
the second strut clamp assembly is securable to the second arm by actuating only the second fastener.

5. The external fixation system of claim 1, wherein the first bone anchoring body comprises a pin clamp body comprising a plurality of pin lock collets, each of which is configured to receive a bone pin and lock the bone pin in place relative to the pin clamp body.

6. The external fixation system of claim 1, wherein the first strut assembly further comprises an adjustment mechanism operable independently of the clutch mechanism to adjust a length of the intermediate portion.

7. The external fixation system of claim 6, wherein:
the intermediate portion comprises:
an outer strut tube comprising an outer strut tube thread; and
an inner strut tube that is slidable within the outer strut tube to adjust the length; and
the adjustment mechanism comprises an adjustment knob rotatably coupled to the inner strut tube, the adjustment knob comprising an inner adjustment knob thread configured to engage the outer strut tube thread to move the outer strut tube axially along the inner strut tube in response to rotation of the adjustment knob.

8. The external fixation system of claim 1, wherein the clutch mechanism is further movable to a locked position in which the clutch mechanism does not allow variation in the length.

9. The external fixation system of claim 1, wherein:
the intermediate portion comprises:
an outer strut tube; and
an inner strut tube that is slidable within the outer strut tube to adjust the length; and
the clutch mechanism comprises:
an adjustment collar with an inner chamfered surface;
a plurality of clutch balls;
a plurality of clutch springs configured to retain the clutch balls between the inner chamfered surface and an outer surface of an inner strut tube; and
a clutch knob that is rotatable to move the clutch balls in relation to the inner chamfered surface such that:
in the restricted position, the inner chamfered surface drives the clutch balls against the outer surface of the inner strut tube in response to reduction in the length; and
in the unlocked position, the inner chamfered surface does not drive the clutch balls against the outer surface of the inner strut tube in response to reduction in the length.

10. The external fixation system of claim 1, further comprising an auxiliary bone anchoring body comprising:
an auxiliary base that is securable at an adjustable location along the length of the intermediate portion; and
an auxiliary pin guide that is polyaxially adjustable relative to the auxiliary base such that the auxiliary pin guide is securable to the first bone at any of a plurality of orientations relative to the auxiliary base.

11. The external fixation system of claim 1, further comprising a second strut assembly comprising:
a third end configured to be removably coupled to the first bone anchoring body;
a fourth end configured to be removably coupled to the second bone anchoring body; and
a second intermediate portion that extends between the third end and the fourth end, the second intermediate portion having a second length that is adjustable.

12. An external fixation system for securing a first bone of a patient relative to a second bone of the patient, the external fixation system comprising:
a first bone anchoring body configured to be secured to the first bone;
a second bone anchoring body configured to be secured to the second bone; and
a first strut assembly comprising:
a first end configured to be removably coupled to the first bone anchoring body;
a second end configured to be removably coupled to the second bone anchoring body;
an intermediate portion that extends between the first end and the second end, the intermediate portion having a length that is adjustable;
an adjustment mechanism operable to adjust a length of the intermediate portion; and
a clutch mechanism operable independently of the adjustment mechanism, wherein the clutch mechanism is movable between:
an unlocked position in which the clutch mechanism allows variation in the length; and
a locked position in which the clutch mechanism does not allow variation in the length.

13. The external fixation system of claim 12, further comprising:
a first strut attachment assembly that is attachable to the first bone anchoring body and to the first end of the first strut assembly; and
a second strut attachment assembly that is attachable to the second bone anchoring body and to the second end of the first strut assembly;
wherein:
the first end of the first strut assembly comprises a first sphere portion;
the second end of the first strut assembly comprises a second sphere portion;
the first bone anchoring body comprises a first arm;
the second bone anchoring body comprises a second arm;
the first strut attachment assembly comprises:
a first fastener configured to secure the first strut attachment assembly to the first arm; and
a first strut clamp assembly comprising a first clamp comprising a first inner clamp surface configured to receive and be tightened around the first sphere portion; and
the second strut attachment assembly comprises:
a second fastener configured to secure the second strut attachment assembly to the second arm; and
a second strut clamp assembly comprising a second clamp comprising a second inner clamp surface configured to receive and be tightened around the second sphere portion;
the first strut clamp assembly is securable to the first arm by actuating only the first fastener; and
the second strut clamp assembly is securable to the second arm by actuating only the second fastener.

14. The external fixation system of claim 12, wherein the first bone anchoring body comprises a pin clamp body comprising a plurality of pin lock collets, each of which is configured to receive a bone pin and lock the bone pin in place relative to the pin clamp body.

15. The external fixation system of claim 12, wherein:
the intermediate portion comprises:
an outer strut tube comprising an outer strut tube thread; and
an inner strut tube that is slidable within the outer strut tube to adjust the length; and
the adjustment mechanism comprises an adjustment knob rotatably coupled to the inner strut tube, the adjustment knob comprising an inner adjustment knob thread configured to engage the outer strut tube thread to move the outer strut tube axially along the inner strut tube in response to rotation of the adjustment knob.

16. The external fixation system of claim 12, wherein the clutch mechanism is further movable to a restricted position in which the clutch mechanism allows one of increase and reduction in the length while restricting the other of increase and reduction in the length.

17. The external fixation system of claim 16, wherein:
the intermediate portion comprises:
an outer strut tube; and
an inner strut tube that is slidable within the outer strut tube to adjust the length; and
the clutch mechanism comprises:
an adjustment collar with an inner chamfered surface;
a plurality of clutch balls;
a plurality of clutch springs configured to retain the clutch balls between the inner chamfered surface and an outer surface of an inner strut tube; and
a clutch knob that is rotatable to move the clutch balls in relation to the inner chamfered surface such that:
in the restricted position, the inner chamfered surface drives the clutch balls against the outer surface of the inner strut tube in response to reduction in the length; and
in the unlocked position, the inner chamfered surface does not drive the clutch balls against the outer surface of the inner strut tube in response to reduction in the length.

18. An external fixation system for securing a first bone of a patient relative to a second bone of the patient, the external fixation system comprising:
a plurality of bone anchoring bodies, each of which is configured to be secured to at least one of the first bone and the second bone, the plurality of bone anchoring bodies comprising:
a first bone anchoring body having a first shape; and
a second bone anchoring body having a second shape different from the first shape;
a strut assembly having a variable length; and
a plurality of strut attachment assemblies, each of which is attachable to any of the bone anchoring bodies and to the strut assembly to secure any of the bone anchoring bodies to the strut assembly.

19. The external fixation system of claim 12, further comprising:
a first strut attachment assembly that is attachable to the first bone anchoring body and to the first end of the first strut assembly; and
a second strut attachment assembly that is attachable to the second bone anchoring body and to the second end of the first strut assembly;
wherein:
the first end of the first strut assembly comprises a first sphere portion;
the second end of the first strut assembly comprises a second sphere portion;
the first bone anchoring body comprises a first arm;
the second bone anchoring body comprises a second arm;
the first strut attachment assembly comprises:
a first fastener configured to secure the first strut attachment assembly to the first arm; and
a first strut clamp assembly comprising a first clamp comprising a first inner clamp surface configured to receive and be tightened around the first sphere portion; and
the second strut attachment assembly comprises:
a second fastener configured to secure the second strut attachment assembly to the second arm; and
a second strut clamp assembly comprising a second clamp comprising a second inner clamp surface configured to receive and be tightened around the second sphere portion;
the first strut clamp assembly is securable to the first arm by actuating only the first fastener; and
the second strut clamp assembly is securable to the second arm by actuating only the second fastener.

20. The external fixation system of claim 18, wherein the first bone anchoring body comprises a pin clamp body comprising a plurality of pin lock collets, each of which is configured to receive a bone pin and lock the bone pin in place relative to the pin clamp body.
